(19) United States
(12) Patent Application Publication
(10) Pub. No.: US 2026/0346288 A1
(43) Pub. Date: Nov. 5, 2026

(54) SYSTEMS AND METHOD FOR MONITORING FLUID IN STORAGE TANKS TO OPERATE PUMPS

(71) Applicant: Nathan Kerley, Marquette Heights, IL (US)

(72) Inventor: Nathan Kerley, Marquette Heights, IL (US)

(21) Appl. No.: 19/661,155

(22) Filed: Apr. 28, 2026

Related U.S. Application Data
(60) Provisional application No. 63/798,387, filed on May 1, 2025.

(51) Int. Cl. B05B 12/08; B05B 9/04; G01N 33/18; G08B 6/00; G08B 21/18

(52) U.S. Cl. CPC B05B 12/081; B05B 9/0403; G01N 33/18; G08B 21/182; G08B 6/00

(57) ABSTRACT

Provided herein are systems controlling pressure washer systems using fluid storage tanks. For example, a system can include a processor configured to obtain sensor data associated with a fluid level measured in a storage tank. The system can compare the fluid level measured in the storage tank with a threshold fluid level representing a minimum level at which a pump can operate safely, and, in response to comparing the fluid level with the threshold fluid level, determine that the fluid level satisfies the threshold fluid level. A device can provide a control signal that is configured to cause the pump to operate based on the fluid level satisfying the threshold fluid level.

### CROSS-REFERENCE TO RELATED APPLICATIONS

This application claims priority to U.S. Provisional Patent Application No. 63/798,387, filed on May 1, 2025, which is incorporated by reference herein in its entirety.

### TECHNICAL FIELD

This application relates generally to systems and methods for controlling pressure washer systems using fluid storage tanks; and, in some embodiments, to systems and methods for monitoring fluid in a fluid storage tank to optimize fluid availability during operation of the system for system cooling.

### BACKGROUND

Pumps (e.g., direct drive pumps, wobble pumps, axial cam pumps, triplex pumps, and/or the like) can be configured to move fluids in various applications (e.g., pressure washing system and/or the like) from one location to another. For example, a direct drive pump can be included in a portable pressure washer to deliver water to the pressure washer. In such an example, the pump can run continuously, driven by sustained operation of a motor. But under certain conditions (e.g., when the fluid source is depleted, when a blockage occurs in an inlet of a fluid storage tank, or to the pump, etc.), the pump can run dry, leading to significant operational challenges. For example, running the pump dry (also referred to as “dry running”) can result in the pump overheating due to the absence of fluid to cool the pump. This can lead to compromised pump performance, mechanical damage, and an overall reduction in the service life of the pump.

### SUMMARY

Further exacerbating these technical issues, there can be difficulty in configuring pump management systems in effectively managing these operational issues. For example, techniques for managing pump operation can involve an individual being present to monitor and manage the pumps. This can cause inefficient pump operation which can lead to the pump entering states where the pump is not cooled.

Some pump management systems include an automatic shutoff feature configured to engage when insufficient fluid is available to the pump. However, these shutoff features can be delayed in engaging, thereby allowing the pump to run dry and overheat, which may result in damage to the pump. Furthermore, conventional pump management systems do not provide real-time fluid-level information to operators. As a result, operators often physically inspect fluid storage tanks to ensure adequate supply, leading to increased labor costs and inefficiencies. These problems are particularly acute in professional pressure-washing operations, where an auxiliary or reserve tank is frequently employed to provide a continuous water supply. Once a reserve tank is depleted, the lack of immediate feedback to the operator can result in pump damage and operational downtime before the operator becomes aware of the low fluid condition.

To overcome this first novel technical problem, in some embodiments, a system is disclosed that monitors fluid in a fluid storage tank to optimize fluid availability during operation of the system for system cooling. The system can include one or more processors. The one or more processors can be configured to obtain sensor data associated with a fluid level measured in a storage tank. The one or more processors can be configured to compare the fluid level measured in the storage tank with a threshold fluid level representing a minimum level at which a pump can operate safely. In response to comparing the fluid level with the threshold fluid level, the one or more processors can be configured to determine that the fluid level satisfies the threshold fluid level. The one or more processors can be configured to provide control data associated with a control signal. The control signal can be configured to cause one or more devices of a pressure washing system to operate based on the fluid level satisfying the fluid level threshold.

By implementing the techniques described herein, systems can be configured to prevent operation of pressure washer systems without an appropriate amount of fluid being available to cool, fill, lubricate, and/or pressurize such systems. For example, systems described herein can be configured to provide control data that is configured to cause one or more devices of a system (e.g., a pressure washing system and/or the like) to operate based on the fluid level of the fluid storage tank satisfying the fluid level threshold. In doing so, the system can identify when the storage tank is about to run out of fluid and, in response, shut off the pumps to prevent inadvertent damage to the system. This, in turn, can reduce the likelihood of one or more components of the system (e.g., the motor, the pump, etc.) overheating. Additionally, the system can obtain a measurement of dissolved solids of the fluid in the fluid storage tank and can provide/present/generate a graphical user interface (GUI), such as in a dashboard on a device remotely located from certain components of the system (e.g., the motor, the pump, etc.), indicating a status of dissolved solids (e.g., that the dissolved solids threshold is satisfied), a status of the fluid (e.g., fluid level, temperature), a status of components of the system (e.g., pump/motor temperature, pressure, flow rate, rpm, “use status” e.g., on or off), and/or any anomaly in system operation (e.g., overheating, significant vibration, or noise). This can improve efficiency as the user does not need to constantly measure or check specific aspects locally, such as the dissolved solids in the fluid which can hinder pressure washer operations. Further, by measuring the amount of dissolved solids, individuals operating such systems can understand with greater accuracy what the concentration of the fluid in the system includes without having to physically monitor and test the concentration. And by implementing the techniques described herein, bandwidth and memory usage can be conserved as only targeted information about the state of the fluid tank is presented to the user operating the pump.

As compared with techniques that involve continuous data transmission between the controller and a device (e.g., a wearable device) to indicate the fluid levels described, the techniques described herein involve the transmission of fewer or discrete sets of data indicating the fluid level of the storage tank (and, in cases where the fluid level satisfies a threshold, generating and transmitting data indicating a warning to the device). This can, in turn, conserve processing and memory resources that would otherwise be dedicated to allowing for the continuous transmission of data to the device. This can also lessen the likelihood of transmission of data using an antenna coupled to the controller interfering with nearby devices. This approach is particularly useful for scalable solutions where multiple tanks and pumps need to be monitored, and individuals operating the pumps can become fatigued or desensitized when constantly reviewing a fluid level to determine whether they are about to cause the pump to run dry.

In an embodiment, a system for monitoring fluid in storage tanks to operate a pump is disclosed. The system can include one or more processors. The one or more processors can be configured to obtain sensor data associated with a fluid level measured in a storage tank. In some implementations, the one or more processors can be configured to compare the fluid level measured in the storage tank with a threshold fluid level representing a minimum level at which a pump can operate safely. In implementations, the one or more processors can be configured to, in response to comparing the fluid level with the threshold fluid level, determine that the fluid level satisfies the threshold fluid level. And in some implementations, the one or more processors can be configured to provide a control signal that is configured to cause the pump to operate based on the fluid level satisfying the threshold fluid level.

In some implementations, the one or more processors can be further configured to: in response to determining that the fluid level satisfies the threshold fluid level, generate a warning via a GUI, the warning indicating that the fluid level in the storage tank is below a safe operation level. In some implementations, the one or more processors can be further configured to render the warning via the GUI at a device.

In some implementations, the one or more processors can be further configured to, in response to determining that the fluid level satisfies the threshold fluid level, generate a warning control signal. In some implementations, the one or more processors can be further configured to provide the warning control signal to a device to initiate operation of a haptic feedback system of the device.

In some implementations, wherein the sensor data can include first sensor data, the one or more processors can be further configured to obtain second sensor data associated with a measurement of dissolved solids in fluid stored in the storage tank. In some implementations, wherein the sensor data can include first sensor data, the one or more processors can be further configured to compare the measurement of the dissolved solids with a dissolved solids threshold to determine that the dissolved solids threshold is satisfied. In some implementations, wherein the sensor data can include first sensor data, the one or more processors can be further configured to in response to determining that the dissolved solids threshold is satisfied, generate an indicator via a GUI, the indicator indicating that the dissolved solids threshold is satisfied; and render the indicator via a GUI of a device.

In some implementations, the one or more processors can be further configured to generate the indicator to indicate a part per million (PPM) level of the dissolved solids in the fluid.

In some implementations, wherein the one or more processors are configured to provide the control signal, the one or more processors can be configured to provide: a sensor signal to a motor indicating low pressure of the fluid, the sensor signal configured to cause the motor coupled to the pump to change operation states.

In some implementations, wherein the one or more processors configured to provide the control signal, the one or more processors can be configured to provide the control signal to a fuel control system to adjust a rate of flow of fuel into the motor from a first rate to a second rate, the second rate associated with an idle state of the motor.

In some implementations, wherein the sensor data can include first sensor data, the one or more processors can be further configured to obtain second sensor data associated with a second fluid level measured in the storage tank at a point in time after the first sensor data is generated. In some implementations, wherein the sensor data can include first sensor data, the one or more processors can be further configured to determine that the second fluid level does not satisfy the threshold fluid level. In some implementations, wherein the sensor data can include first sensor data, the one or more processors are configured to cause the fuel control system to adjust the rate of flow of fuel into the motor, from the second rate to the first rate.

In an embodiment, a method is disclosed. The method can include one or more processors. The one or more processors can be configured to obtain sensor data associated with a fluid level measured in a storage tank. In some implementations, the one or more processors can be configured to compare the fluid level measured in the storage tank with a threshold fluid level representing a minimum level at which a pump can operate safely. In some implementations, in response to comparing the fluid level with the threshold fluid level, the one or more processors can be configured to determine, that the fluid level satisfies the threshold fluid level. In some implementations, the one or more processors can be configured to provide a control signal that is configured to cause the pump to operate based on the fluid level satisfying the threshold fluid level.

In some implementations, in response to determining that the fluid level satisfies the threshold fluid level, the one or more processors can be configured to generate a warning via a GUI, the warning indicating that the fluid level in the storage tank is below a safe operation level. The one or more processors can be configured to render the warning via a GUI of a device.

In some implementations, in response to determining that the fluid level satisfies the threshold fluid level, the one or more processors can be configured to generate a warning control signal. The one or more processors can be configured to provide the warning control signal to a device to initiate operation of a haptic feedback system of the device.

In some implementations, wherein the sensor data can include first sensor data, the one or more processors can be configured to obtain second sensor data associated with a measurement of dissolved solids in fluid stored in the storage tank. The one or more processors can be configured to compare the measurement of the dissolved solids with a dissolved solids threshold to determine that the dissolved solids threshold is satisfied. The one or more processors can be configured to, in response to determining that the dissolved solids threshold is satisfied, generate an indicator via a GUI indicating that the dissolved solids threshold is satisfied; and rendering the indicator via the GUI of a device.

In some implementations, wherein generating the GUI can include the one or more processors can be configured to generate the GUI to indicate a part per million (PPM) level of the dissolved solids in the fluid. In some implementations, wherein generating the GUI can include the one or more processors can be configured to cause the GUI to be displayed, the one or more processors can be configured to provide GUI data associated with the GUI to a device that is configured to receive the GUI data and generate a user interface in accordance with the GUI.

In some implementations, wherein providing control signal can include, the one or more processors configured to provide a sensor signal to a motor indicating low pressure of fluid, the sensor signal configured to cause the motor coupled to the pump to change operation states.

In some implementations, wherein providing control signal can include the one or more processors configured to provide the control signal to a fuel control system to adjust a rate of flow of fuel into a motor to transition from a first rate to a second rate, the second rate associated with an idle state of the motor.

In some implementations, wherein the sensor data can include first sensor data, can include the one or more processors configured to obtain second sensor data associated with a second fluid level measured in the storage tank at a point in time after the first sensor data is generated. In some implementations, wherein the sensor data can include first sensor data, can include the one or more processors configured to determine that the second fluid level does not satisfy the threshold fluid level. In some implementations, wherein the sensor data can include first sensor data, can include the one or more processors configured to provide a second control signal that is configured to cause the fuel control system to adjust the rate of flow of fuel into the motor to transition from the second rate to the first rate.

In yet another embodiment, a non-transitory computer-readable medium is disclosed as storing instructions thereon that, when executed by one or more processors, cause the one or more processors to: obtain sensor data associated with a fluid level measured in a storage tank. The instructions can cause the one or more processors to compare the fluid level measured in the storage tank with a threshold fluid level representing a minimum level at which a pump can operate safely. The instructions can cause the one or more processors to, in response to comparing the fluid level with the threshold fluid level, determine that the fluid level satisfies the threshold fluid level; and provide control data associated with a control signal that is configured to cause the pump to operate based on the fluid level satisfying the threshold fluid level.

In at least some aspects, wherein the instructions further can cause the one or more processors to: in response to determining that the fluid level satisfies the threshold fluid level, generate a warning via a GUI, the warning indicating that the fluid level in the storage tank is below a safe operation level. The instructions can further cause the one or more processors to render the warning via GUI of a device.

In at least some aspects, wherein the instructions further can cause the one or more processors to, in response to determining that the fluid level satisfies the threshold fluid level, generate a warning control signal. The instructions can further cause the one or more processors to, in response to determining that the fluid level satisfies the threshold fluid level, provide the warning control signal to a device to initiate operation of a haptic feedback system of the device.

In at least some aspects, the instructions can further cause the one or more processors to obtain second sensor data associated with a measurement of dissolved solids in fluid stored in the storage tank, wherein the sensor data can include first sensor data. The instructions can further cause the one or more processors to compare the measurement of the dissolved solids with a dissolved solids threshold to determine that the dissolved solids threshold is satisfied. The instructions can further cause the one or more processors to generate an indicator via a GUI, the indicator indicating that the dissolved solids threshold is satisfied, in response to determining that the dissolved solids threshold is satisfied. The instructions can further cause the one or more processors to render the indicator via a GUI of a device.

Additionally or alternatively in pressure washing systems, chemicals or detergents can be mixed with fluids, and pumps can be implemented by these systems to move the fluids toward a targeted surface. In many examples, motors operate these pumps to efficiently move the fluids from one location to another and can be adjusted by a throttle that controls the energy delivered to the motor (e.g., fuel flow, etc.) to increase or decrease the speed at which the motor is operating. However, pressure washing systems can run inefficiently when used for various tasks, resulting in mechanically inefficient operation and premature wear on the components of the pump. For example, users can operate pressure washing systems that apply a constant load to a motor, and, over time, the pump can slow due to wear caused by friction. As a result, the pump may operate at a reduced flow rate that is inadequate for the various tasks being performed (e.g., cleaning). Because these systems are not configured for dynamic adjustment of the rate at which the pump is operated, particularly when users are at a distance from the system and cannot reach and/or see the system, this mechanical wear can continue unmitigated, resulting in an overall reduction in the service life of the motor and corresponding reductions in performance by the pump.

As described herein, a controller can be implemented by pressure washing systems to address certain issues that cause degraded performance. However, some challenges to these systems still include achieving real-time monitoring and control of the motor speed to maintain a desired amount of pressure generated by the pump. For example, a controller that causes the application of a constant load or throttle to the pump during operation of the pressure washing system may not address the problem of the pump slowing down over time due to variations in load on the pump, friction wearing various components of the pump and/or motor, etc. This can result in repeated manual adjustment of the controller to maintain a desired pressure (e.g., through adjustment of the pressure output), which in some cases results in wasting the fluid being dispersed using the pump.

To overcome this second novel technical problem, the techniques described herein relate to a system for controlling a motor by dynamically adjusting operation of an energy delivery system to maintain a set speed of the motor (e.g., rotations per minute or RPMs). The system can include one or more processors configured to, in response to obtaining first monitor data associated with a first speed of a motor, compare the first speed to a hold speed to determine a first difference. In some examples, the hold speed can be set based on input received by a user. The system can then determine a throttle setting based on the first difference to adjust operation of the motor and reduce the first difference. In examples, the system can provide a first control signal to a throttle system to adjust operation of the throttle system based on the throttle setting and, in response to providing the first control signal, obtain second monitor data associated with a second speed of the motor. The system can then compare the second speed to the hold speed to determine a second difference and provide a second control signal based on the second difference. This control signal can cause adjusted operation of the motor, resulting in an increase or decrease in RPMs. And in some examples, this process can be performed iteratively (e.g., periodically or continuously) to maintain desired performance (e.g., fluid flow at various pressures) at the pump.

By implementing the techniques described herein, systems can be configured by a user to operate the pressure washer systems at a set speed of the motor and thereby achieve a consistent fluid flow rate over time, despite the wear experienced by the pump or changes in load on the motor controlling the pump. By implementing a controller to maintain operation of the pump at the set speed (e.g., in response to user input), the flow rate at which the pump is operated can be precisely controlled, increasing the efficiency and precision of the pressure washing process. For example, in the case where a lower speed and flow rate is optimal, such as a user changing from cleaning a faraway surface to a nearby one, a lower hold speed can be set by the user to adjust the motor speed and corresponding fluid flow rate. Operating the pressure washing system at a precise flow rate for a task result in better performance of the pressure washing system and prevents excessive use of resources (e.g., water, gas, chemical) and mechanical wear on the motor.

Furthermore, the system can achieve improvements over conventional systems by selectively controlling whether a control signal is generated to adjust operation of the throttle system. This can allow for fewer changes to energy delivered to the motor than would be made during continuous adjustment of the operation of the system, resulting in less frequent adjustment to the motor's operation to maintain a set motor speed. This in turn reduces the computational load as control signals are only generated periodically resulting in fewer control signals generated over time.

The system described can be compatible with a variety of motors and throttles, which have various properties, including a range of throttle positions. For example, a system can be configured to set throttle positions based on (e.g., after) being calibrated to a motor having a predetermined configuration (e.g., a predetermined model). This can allow the system to automate adjustment of energy to the motor and, as a result, improve precision of the control of the pressure washing process. Compared with systems that operate based solely on manual control by a user, the systems described herein can be calibrated to protect the motor and throttle from damage from exceeding their operational range while also maintaining flow rates as desired over time.

In some implementations, a system is described for controlling a motor by dynamically adjusting operation of an energy delivery system to maintain a set number of rotations per minute in accordance with a set point. The system can include one or more processors configured to: in response to obtaining first monitor data associated with a first speed of a motor, compare the first speed to a hold speed to determine a first difference. The hold speed can be set based on input received by a user. In examples, the one or more processors can be configured to determine a throttle setting based on the first difference to adjust operation of the motor and reduce the first difference. The one or more processors can then provide a first control signal to a throttle system to adjust operation of the throttle system based on the throttle setting. In response to providing the first control signal, the one or more processors can obtain second monitor data associated with a second speed of the motor. The one or more processors can then compare the second speed to the hold speed to determine a second difference and provide a second control signal to adjust operation of the throttle system based on the second difference.

In some implementations, a system is described, where the one or more processors can be further configured to obtain input data associated with an input received at a user device indicating the hold speed. The one or more processors configured to compare the first speed of the motor to the hold speed can be configured to compare the first speed of the motor to the hold speed in response to receiving the input data.

In some implementations, a system is described, wherein the one or more processors configured to determine the throttle setting to adjust operation of the motor can be configured to compare the first difference to one or more threshold differences to select a threshold difference. Each threshold difference can be associated with increases or decreases of energy to the motor. The one or more processors can determine an increase or decrease of energy to the motor based on the threshold difference. The one or more processors can then determine the throttle setting to adjust operation of the motor based on the increase or decrease of energy to the motor.

In some implementations, a system, wherein the one or more processors can be further configured to generate an indicator to indicate a rotations per minute (RPM) value associated with the hold speed. In some examples, the one or more processors can be configured to generate the indicator via a graphical user interface (GUI) on a user device. The one or more processors can then render the indicator using a display device of the user device.

In some implementations, a system is described, wherein the one or more processors can be further configured to in response to obtaining monitor data associated with the first speed or the second speed of the motor, generate an indicator to indicate an RPM value associated with the first speed or the second speed. In some examples, the one or more processors can be configured to generate the indicator via a graphical user interface (GUI) on a user device. The one of more processors can then render the indicator using a display device of the user device.

In some implementations, a system is described, wherein the one or more processors can be further configured to receive user input data associated with a plurality of user inputs indicating positions of the throttle system to determine a set of calibration throttle settings. In response to receiving the plurality of user inputs indicating the positions of the throttle system, the one or more processors can be further configured to obtain a plurality of motor sensor signals associated with a plurality of sensed positions of the throttle system. The one or more processors can configure the set of calibration throttle settings based on the plurality of user inputs indicating the positions of the throttle system and the plurality of sensed positions of the throttle system. The one or more processors can then calibrate the throttle setting based on the first difference to adjust operation of the motor to reduce the first difference with the set of calibration throttle settings.

In some implementations, a system is described, wherein a first position in the plurality of user inputs indicating positions can correspond to a position where the throttle system can be at its widest angle. And a second position in the plurality of user inputs indicating positions can correspond to a position where the throttle system can be at its narrowest angle.

In some implementations, a system is described, wherein the motor can include a first motor. The one or more processors can be further configured to determine that the first motor can be associated with a first model that was used to calibrate the throttle setting. In response to obtaining an indication that the first motor was replaced with a second motor, the one or more processors can determine that at least one previous set of calibration throttle settings for the first model can be associated with the second motor. If at least one previous set of calibration throttle settings satisfies this criteria, the one or more processors can then retrieve a previous set of calibration throttle settings associated with the first model and configure the set of calibration throttle settings to the previous set of calibration throttle settings.

In some implementations, a method is described for controlling a motor by dynamically adjusting operation of an energy delivery system to maintain a set number of rotations per minute in accordance with a set point. The method can include one or more processors. In some examples, the one or more processors, can in response to obtaining first monitor data associated with a first speed of a motor, compare the first speed to a hold speed to determine a first difference. The hold speed can be set based on input received by a user. In embodiments, the one or more processors can be configured to determine a throttle setting based on the first difference to adjust operation of the motor and reduce the first difference. In some examples, the one or more processors can be configured to provide a first control signal to a throttle system to adjust operation of the throttle system based on the throttle setting. In response to providing the first control signal, the one or more processors can be configured to obtain second monitor data associated with a second speed of the motor. In an example, the one or more processors can be configured to compare the second speed to the hold speed to determine a second difference. Then for example, the one or more processors can be configured to provide a second control signal to adjust operation of the throttle system based on the second difference.

In some implementations, a method is further described including that in examples, the one or more processors can be configured to obtain input data associated with an input received at a user device indicating the hold speed. In some embodiments, the one or more processors can be configured to compare the first speed of the motor to the hold speed in response to receiving the input data.

In some implementations, a method is further described including that in some implementations, the one or more processors can be configured to determine the throttle setting to adjust operation of the motor including comparing the first difference to one or more threshold differences to select a threshold difference. Each threshold difference can be associated with increases or decreases of energy to the motor. In some examples, the one or more processors can be configured to determine an increase or decrease of energy to the motor based on the threshold difference. Then, in some embodiments, the one or more processors can be configured to determine the throttle setting to adjust operation of the motor based on the increase or decrease of energy to the motor.

In some implementations, a method is further described including that in some examples, the one or more processors can be configured to generate an indicator to indicate a rotations per minute (RPM) value associated with the hold speed via a graphical user interface (GUI) on a user device. Then, the one or more processors can be configured to render the indicator using a display device of the user device.

In some implementations, a method is further described including that in some embodiments, in response to obtaining monitor data associated with the first speed or the second speed of the motor, the one or more processors can be configured to generate an indicator to indicate an RPM value associated with the first speed or the second speed via a graphical user interface (GUI) on a user device. Then, in some examples, the one or more processors can be configured to render, the indicator using a display device of the user device.

In some implementations, a method is further described including that in some examples, the one or more processors can be configured to receive user input data associated with a plurality of user inputs indicating positions of the throttle system to determine a set of calibration throttle settings. In response to receiving the plurality of user inputs indicating the positions of the throttle system, the one or more processors can be configured to obtain a plurality of motor sensor signals associated with a plurality of sensed positions of the throttle system. In examples, the one or more processors can be configured to configure the set of calibration throttle settings based on the plurality of user inputs indicating the positions of the throttle system and the plurality of sensed positions of the throttle system. Then, in some embodiments, the one or more processors can be configured to calibrate the throttle setting based on the first difference to adjust operation of the motor to reduce the first difference with the set of calibration throttle settings.

In some implementations, a method is further described, wherein a first position in the plurality of user inputs indicating positions corresponds to a position where the throttle system can be at its widest angle. And a second position in the plurality of user inputs indicating positions corresponds to a position where the throttle system can be at its narrowest angle.

In some implementations, a method is further described, wherein the motor includes a first motor. In some examples, the one or more processors can be configured to determine that the first motor can be associated with a first model that was used to calibrate the throttle setting. In response to obtaining an indication that the first motor was replaced with a second motor, the one or more processors can be configured to determine that at least one previous set of calibration throttle settings for the first model can be associated with the second motor. If at least one previous set of calibration throttle settings satisfies this criteria, the one or more processors can be configured to retrieve a previous set of calibration throttle settings associated with the first model and configure the set of calibration throttle settings to the previous set of calibration throttle settings.

In some implementations, a non-transitory computer-readable medium is described, storing instructions there on that, when executed by one or more processors, can cause the one or more processors to, in response to obtaining first monitor data associated with a first speed of a motor, compare the first speed to a hold speed to determine a first difference. The hold speed can be set based on input received by a user. In some examples, the instructions, when executed by one or more processors, can determine a throttle setting based on the first difference to adjust operation of the motor and reduce the first difference. In embodiments, the instructions, when executed by one or more processors, can provide a first control signal to a throttle system to adjust operation of the throttle system based on the throttle setting. In response to providing the first control signal, the instructions, when executed by one or more processors, can obtain second monitor data associated with a second speed of the motor. In some examples, the instructions, when executed by one or more processors, can then compare the second speed to the hold speed to determine a second difference, and provide a second control signal to adjust operation of the throttle system based on the second difference.

In some implementations, a non-transitory computer-readable medium is described, wherein the instructions further can cause the one or more processors to obtain input data associated with an input received at a user device indicating the hold speed. The one or more processors configured to compare the first speed of the motor to the hold speed can be configured to compare the first speed of the motor to the hold speed in response to receiving the input data.

In some implementations, a non-transitory computer-readable medium is described, wherein the instructions further can cause the one or more processors to compare the first difference to one or more threshold differences to select a threshold difference. Each threshold difference can be associated with increases or decreases of energy to the motor. In some examples, the instructions, when executed by one or more processors, can determine an increase or decrease of energy to the motor based on the threshold difference. In embodiments, the instructions, when executed by one or more processors, can then determine the throttle setting to adjust operation of the motor based on the increase or decrease of energy to the motor.

In some implementations, a non-transitory computer-readable medium is described, wherein the sensor data can include first sensor data, and the instructions can further cause the one or more processors to generate an indicator to indicate a rotations per minute (RPM) value associated with the hold speed via a graphical user interface (GUI) on a user device. In some embodiments, the instructions, when executed by one or more processors, can then render the indicator using a display device of the user device.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates a diagram of a system for monitoring fluid in a fluid storage tank to optimize fluid availability during operation of the system for system cooling, according to an embodiment.

FIG. 2 illustrates a flow diagram of a process for monitoring fluid in a fluid storage tank to optimize fluid availability during operation of the system for system cooling, according to an embodiment.

FIG. 3 illustrates an example schematic block diagram of a computing system, according to an embodiment.

FIG. 4-8 illustrate example implementations of a user interfaces (UIs) displayed on the device, according to an embodiment.

FIG. 9 illustrates an example implementation of a printed circuit board (PCB), according to an embodiment.

FIG. 10 illustrates a diagram of a system for controlling a motor by dynamically adjusting operation of an energy delivery system to maintain a set number of rotations per minute in accordance with a set point, in accordance with one or more embodiments.

FIG. 11 illustrates a flow diagram of a process for controlling a motor by dynamically adjusting operation of an energy delivery system to maintain a set number of rotations per minute in accordance with a set point, according to an embodiment.

FIG. 12 illustrates an example implementation of a user device, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are configured to be considered within the scope of the subject matter disclosed herein. Other embodiments can be used, or other changes can be made without departing from the spirit or scope of the present disclosure. The illustrative embodiments described in the detailed description are not meant to be limiting of the subject matter presented.

In examples described herein, systems for monitoring fluid in storage tanks to operate a pump can include one or more processors configured to obtain sensor data associated with a fluid level measured in a storage tank. The processors can compare the fluid level measured in the storage tank with a threshold fluid level representing, for example, a minimum level at which a pump can operate safely. In response to comparing the fluid level with the threshold fluid level, the processors can determine that the fluid level satisfies the threshold fluid level and can provide a control signal that is configured to cause the pump to operate based on the fluid level satisfying the threshold fluid level.

In some embodiments, the systems described herein can include a custom PCB with a Long-Range (LoRa) radio transmitter, relays, sensors, and a user-wearable device. The systems described can include one or more (e.g., two) sensors installed on or in a buffer tank. These sensors can include an ultrasonic sensor that can measure water levels and a photoelectric sensor that can act as a failsafe by shutting off pressure washing equipment when water levels drop too low (e.g., below a threshold value). As an example, the ultrasonic sensor can be configured to measure water levels in the storage tank within a first range, and the photoelectric sensor can be configured to measure water levels within a second range that is lower than the first range. A wearable device such as a smart watch with a printed circuit board (PCB) and LoRa can allow operators to monitor water levels, receive alerts, and control various functions such as fuel levels, electric pumps, total dissolved solids (TDS) (e.g., for window cleaning, house cleaning, etc.), pressure washer settings, motor operations, etc. These functions can be initiated at the wearable device (e.g., on the wrist of the operator) and from a distance of, for example, three hundred feet from a storage/buffer tank. Additional modules can include a TDS monitor to check the total dissolved solids in water for window cleaning, a voltage monitor to display the current voltage of multiple 12v DC batteries on the wearable device, a soft start module to control the engagement of an electromagnetic clutch based on pressure buildup in the system, an engine start/stop module to remotely control the pressure washing motors, and a system monitoring module to oversee the entire system, allowing users to add or remove modules and review system performance.

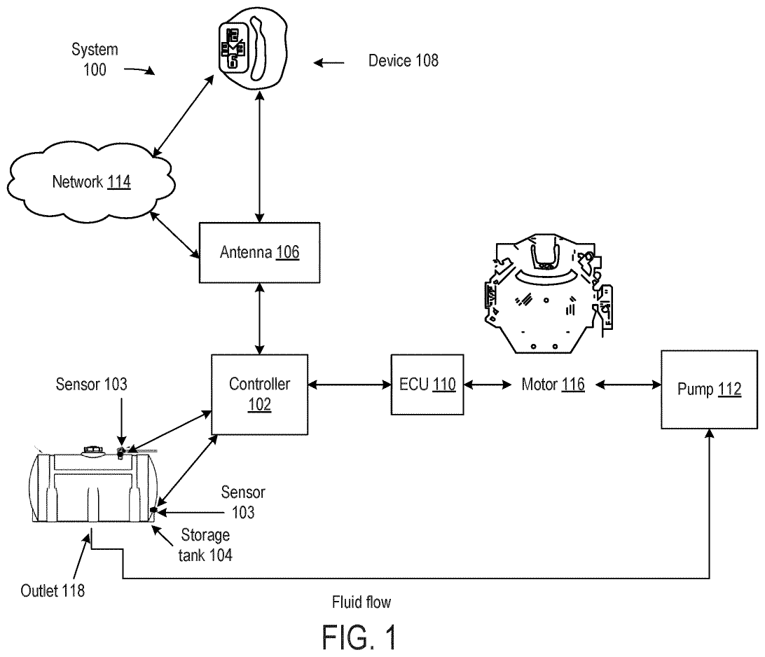

FIG. 1 illustrates components of a system 100 for monitoring fluid in a fluid storage tank to optimize fluid availability during operation of the system for system cooling, according to an embodiment. The system 100 can include a controller 102, a storage tank 104 (also referred to as a fluid tank), an antenna 106, a device 108, an ECU 110, a pump 112, a network 114, a motor 116, and an outlet 118. Various components depicted in FIG. 1 can be included in a pressure washer system which individuals operate to clean various objects (e.g., structures, buildings, etc.) that includes one or more pressure washer pumps (e.g., the pump 112). While the system 100 is shown as including various components, it will be understood that similar systems are not confined to the components described herein and can include additional or other components, not shown for brevity.

The controller 102 can include any appropriate logic device (e.g., processing device, microcontroller, processor, application-specific integrated circuit (ASIC), field programmable gate array (FPGA), memory storage device, memory reader, or other device or combinations of devices) that can be adapted to execute, store, and/or receive appropriate instructions, such as software instructions implementing a fluid monitoring system for controlling various operations of a pressure washer system such as system 100. Such software instructions can also implement methods for obtaining sensor data, processing sensor 103 information, determining sensor 103 information, providing control signals (e.g., through the controller 102), generating warning control signals, or performing any of the various operations described herein. For example, the controller 102 can include any portion of the ECU 110.

A storage tank 104 can include any container that can be adapted to hold various fluids, such as water, water with a substance suspended or diluted (such as solvents) therein, etc. Examples of storage tanks can include a buffer tank, water tank, reservoirs, cylinders, stainless steel tanks, fiberglass tanks, collapsible water bladders, polyethylene tanks, etc. The storage tank 104 can include one or more sensors 103 disposed therein and/or thereon. The one or more sensors 103 can include ultrasonic sensors, photoelectric sensors, temperature sensors, float switches, magneto strictive sensors, capacitive sensors, radar sensors, conductive sensors, etc. The one or more sensors 103 can be placed along, on, inside or near the storage tank 104. The storage tank 104 can be connected to the controller 102 using one or more wired or wireless connections as described herein. In some embodiments, the storage tank can have a fluid inlet or an outlet 118. For example, the storage tank can have a fluid inlet that is configured to receive fluid from an external source such as water from a water source. In this example, the storage tank can have the outlet 118, where the outlet 118 is configured to couple to the pump 112 using one or more hoses, etc.

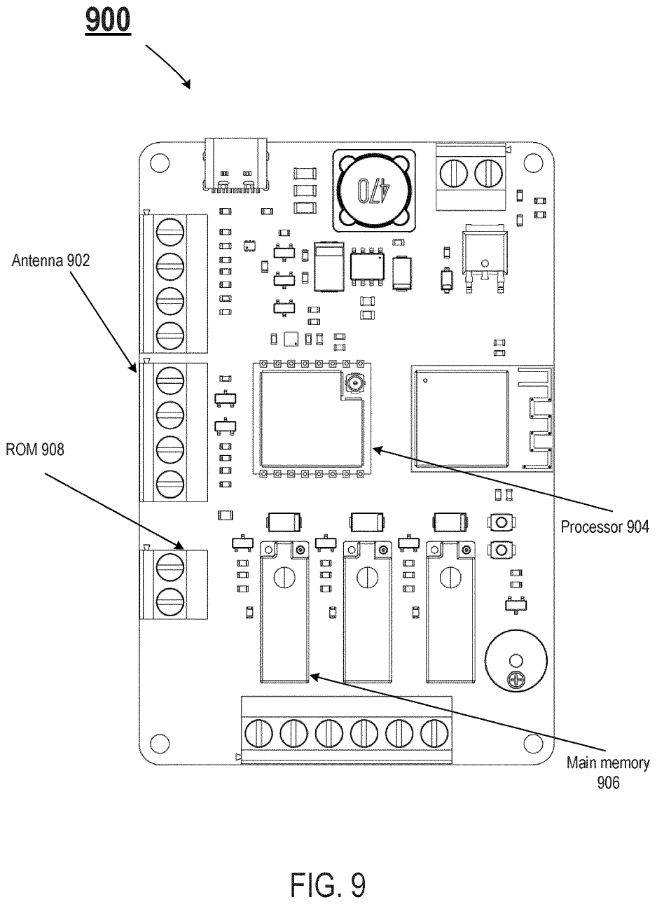

The antenna 106 can include any component or assembly capable of transmitting and/or receiving electromagnetic signals as described herein. For example, the antenna 106 can include a monopole antenna, dipole antenna, patch antenna, slot antenna, loop antenna, helical antenna, fractal antenna, microstrip antenna, or phased array antenna. For example, the antenna 106 can be integrated into or mounted on a device, such as a printed circuit board (PCB) (e.g., the PCB 900 of FIG. 9), a flexible substrate, or an external enclosure. The antenna 106 can be capable of supporting the various communication protocols, frequency ranges, and applications described herein, including, but not limited to, wireless communication systems (e.g., Wi-Fi, Bluetooth, cellular networks), positioning systems (e.g., GPS), or specialized applications such as radar, near-field communication (NFC), or energy harvesting. The antenna 106 can have an omnidirectional or directional radiation pattern. The antenna 106 can transmit signals to and from the device 108 via network 114. The antenna 106 can be connected to the controller 102.

The device 108 can be any computing device, including a processor and a non-transitory machine-readable storage medium capable of performing the various tasks and processes described herein. Examples of a device 108 can include a smart watch, workstation computer, laptop computer, tablet computer, smart phone, or server computer. In operation, various users such as individuals operating pressure washers can access the GUI managed by the devices 108 to monitor the storage tank 104 and the pump 112.

The ECU 110 can include any computing device comprising a processor and a non-transitory machine-readable storage medium capable of performing the various tasks and processes described herein. For example, the ECU 110 can include a microcontroller unit, an embedded system, a dedicated computing device, a programmable logic controller, an engine control module (ECM), and a central processing unit (CPU). In some embodiments, the ECU 110 can be managed by the controller 102 to control the pump 112. In some embodiments, the ECU 110 can control the pump 112. In some embodiments, the ECU 110 can control the motor 116.

The pump 112 can include any device or system configured to move fluids, gases, or other flowable materials through a conduit or system as described herein. For example, the pump 112 can include a triplex plunger pump, an axial cam pump, a direct drive pump, a belt drive pump, or a gear drive pump. The pump 112 can pump fluid from the storage tank 104 to one or more other components of the system 100. In some embodiments, the ECU 110 can manage the pump 112 by sending signals to pump 112.

The motor 116 can include a device or system configured to power or cause movement of various components or systems as described herein. For example, the motor 116 can include electric motors, internal combustion motors, gas turbines, and compression ignition motors. The motor 116 can be coupled to the pump 112 to mechanically engage the pump 112 and cause the pump to obtain fluid from the storage tank 104 and move the fluid toward or through to one or more components of a pressure washer device.

The outlet 118 can be a device, system, or part of the storage tank 104 configured to receive or output fluid from a tank containing fluid, such as the storage tank 104. For example, the outlet 118 can include or be a port, valve, or other fluid-transfer component that enables fluid to flow from the storage tank 104 to a pump or other receiving system. In some embodiments, the outlet 118 may be in fluid communication with a pipe, hose, or manifold assembly configured to deliver fluid from the storage tank 104 to one or more components of downstream equipment (e.g., a pressure washer wand, etc.).

At least some of the above-mentioned components can be connected to each other through a network 114. Examples of the network 114 can include, but are not limited to, private or public local-area-networks (LAN), wireless LAN (WLAN) networks, the Internet, etc. The network 114 can include wired or wireless communications according to one or more standards or via one or more transport mediums. The communication over the network 114 can be performed in accordance with various communication protocols such as Transmission Control Protocol and Internet Protocol (TCP/IP), User Datagram Protocol (UDP), and IEEE communication protocols. In one example, the network 114 can include wireless communications according to Bluetooth specification sets or another standard or proprietary wireless communication protocol. In another example, the network 114 can also include communications over a cellular network, including, e.g., a GSM (Global System for Mobile Communications), CDMA (Code Division Multiple Access), and EDGE (Enhanced Data for Global Evolution) network.

With continued reference to FIG. 1, the controller 102 can obtain sensor data associated with a fluid level measured in a storage tank 104 by a sensor 103. For example, the controller 102 can obtain sensor data associated with a fluid level, where the sensor data is indicative of the fluid level, a temperature of the fluid, or an amount of fluid in the storage tank 104. In an example, the controller 102 can obtain sensor data from at least one sensor 103 located in or on the storage tank 104. There can be multiple sensors 103 in or on the storage tank 104 that measure the fluid level in the storage tank 104. The sensors 103 can include ultrasonic sensors, photoelectric sensors, float switches, temperature sensors, capacitive sensors, conductive sensors, magneto strictive sensors, and radar sensors.

In some embodiments, the storage tank 104 can include an ultrasonic sensor 103 that measures the fluid level in the storage tank 104 at a predetermined rate (e.g., every second) and sends data associated with the fluid level to the controller 102. In this example, the ultrasonic sensor can be placed at the top of the storage tank 104 and be pointing substantially downward towards the bottom of the storage tank 104. For example, the storage tank 104 can include a photoelectric sensor placed one (1) inch above the outlet 118 of the storage tank 104. In this example, the photoelectric sensor can send a signal to the controller 102 when the fluid level drops below the photoelectric sensor. For example, the sensor data associated with a fluid level measured in a storage tank 104 can include the proportion of the storage tank 104 occupied by the fluid. In this example, the fluid level can include ten percent of the storage tank 104 capacity. The fluid level can include a measurement of twenty liters, three gallons, or six cups. The fluid level can include the distance of the fluid above the outlet 118 of the storage tank 104 the fluid level is. In this example, the fluid level can be one inch, one foot, etc., above the outlet 118 of the storage tank 104.

In some embodiments, the controller 102 can obtain second sensor data associated with a measurement of dissolved solids in fluid stored in the storage tank 104. For example, the controller 102 can obtain second sensor data associated with a measurement of dissolved solids in fluid stored in the storage tank 104, where the second sensor data is indicative of a parts per million (PPM) measurement of dissolved solids in fluid. In this example, the measurement of the dissolved solids can include ten parts per million (PPM). For example, the measurement of the dissolved solids can include one (1) liter. The dissolved solids in fluid can include or be window washing chemicals, antifreeze, detergents, degreasers, acids, bleaching agents, alkaline cleaners, etc. For example, the controller 102 can obtain the second sensor data associated with a measurement of dissolved solids in fluid stored in the storage tank 104 using sensors 103 located in the storage tank 104. In this example, the sensors 103 can include total dissolved solids sensors, conductivity sensors, optical sensors, ion-selective electrodes sensors, or capacitive sensors.

In some embodiments, the controller 102 can obtain second sensor data associated with a second sensor fluid level measured in the storage tank 104 at a point in time after the first sensor data is generated. For example, the controller 102 can obtain second sensor data associated with a fluid level, where the second sensor data is indicative of the fluid level, a temperature of the fluid, or an amount of fluid in the storage tank 104. For example, the controller 102 can obtain second sensor data from at least one sensor 103 located in or on the storage tank 104. For example, the second sensor data can be obtained one (1) minute, ten (10) minutes, one (1) second, one (1) hour, five (5) minutes, or twenty (20) minutes after the first sensor data is generated. For example, the second fluid level can be or include the fluid level. The second sensor fluid level can indicate a measurement that is lower or higher than the fluid level. For example, the second sensor data associated with a second sensor fluid level measured in the storage tank 104 can include the proportion of the storage tank 104 occupied by the fluid. In this example, the fluid level can include ten percent of the storage tank 104 capacity. For example, the second fluid level can include a measurement of twenty liters, three gallons, or six cups. In this example, at least one sensor 103 is located in the storage tank 104 and can be located at the bottom of the storage tank 104. For example, the controller 102 can obtain second sensor data within ten seconds, seven minutes, or fifteen minutes after the first sensor data is generated. For example, the second fluid level can include how far above the outlet 118 of the storage tank 104 the second fluid level is. In this example, the second fluid level can be one inch, etc., above the outlet 118 of the storage tank 104.

In some embodiments, the controller 102 can compare the fluid level measured in the storage tank 104 with a threshold fluid level representing a minimum level at which the pump 112 can operate safely. For example, the controller 102 can compare the fluid level measured in the storage tank 104 with a threshold fluid level representing a minimum level at which the pump 112 can operate. For example, the controller 102 can compare the fluid level measured in the storage tank 104 with a threshold fluid level representing a maximum fluid capacity of the storage tank 104. For example, the controller 102 can compare the fluid level measured in the storage tank 104 with a threshold fluid level representing a minimum level at which the pump 112 can operate. The threshold fluid level representing a minimum level at which a pump can operate safely can include at least one inch of fluid above the outlet 118 of the storage tank 104, five gallons of fluid, ten percent of storage tank 104 capacity, or ten inches of fluid. For example, the fluid level can include the fluid in the storage tank 104. For example, the pump 112 can operate safely when there is at least two gallons of water present in the storage tank 104. For example, the pump 112 can operate safely when there is enough fluid in the storage tank 104 to fully engage the pump 112. For example, the pump 112 can operate safely when there is sufficient fluid to cool the pump 112. For example, the pump 112 can operate safely when the pump 112 is functioning at its maximum volumetric capacity.

In some embodiments, the controller 102 can compare the measurement of the dissolved solids with a dissolved solids threshold to determine that the threshold dissolved solids level is satisfied. For example, the threshold dissolved solids level can be satisfied when the dissolved solids measurement is below ten parts per million. The threshold dissolved solids level can be a measurement of parts per million of window washing fluid. For example, the threshold dissolved solids level can include a part per million measurement of the fluid and a temperature of the fluid.

In some embodiments, in response to comparing the fluid level with the threshold fluid level, the controller 102 can determine that the fluid level satisfies the threshold fluid level. For example, the controller 102 can determine that the fluid level satisfies the threshold fluid level by receiving data from the sensors 103 located in the storage tank 104 indicating that the fluid level of the storage tank 104 is less than one inch above the outlet 118 of the storage tank 104, the volume of fluid in the storage tank 104 is less than three gallons, or the height of the fluid in the storage tank 104 is below five inches, and determining that an amount of fluid in the storage tank satisfies the threshold fluid level. The determination that the fluid level satisfies the threshold fluid level can include when the fluid level is above the outlet 118 of the storage tank 104. For example, the controller 102 can integrate data from multiple sensors 103 to provide a more accurate assessment of the fluid level, considering factors such as fluid temperature and pressure.

In some embodiments, in response to determining that the fluid level satisfies the threshold fluid level, the controller 102 can generate at least one warning via graphical user interface (GUI), the warning indicating that the fluid level in the storage tank 104 is below a safe operation level. For example, the fluid level in the storage tank 104 is below a safe operation level when the fluid level reaches one inch above the outlet 118 of the storage tank 104, the volume of fluid in the storage tank 104 is more than three gallons, or the height of the fluid in the storage tank 104 is above five inches. For example, the warning via graphical user interface (GUI) can indicate that the fluid level is one inch above the outlet 118 of the storage tank 104, the volume of fluid in the storage tank 104 is more than three gallons, or the height of the fluid in the storage tank 104 is above five inches. For example, the warning via graphical user interface (GUI) can indicate that the fluid level of the storage tank 104 is low or that the fluid level of the storage tank 104 is exceeding the fluid capacity of the storage tank 104, and that the fluid level satisfies the threshold fluid level. For example, the warning via graphical user interface (GUI) can include a flashing screen, a blinking icon, an icon changing color, or a blinking screen. In this example, the flashing screen can flash repeatedly or once. While certain concepts of the present disclosure are discussed with respect to specific threshold values, it will be understood that any suitable threshold value can be implemented by the systems described herein.

In some embodiments, in response to determining that the fluid level satisfies the threshold fluid level, the controller 102 can generate a warning control signal. For example, the controller 102 can generate at least one warning control signal for the device 108 or the ECU 110. In this example, the warning signal can indicate that the fluid level of the storage tank 104 is low or that the fluid level of the storage tank 104 is exceeding the fluid capacity of the storage tank 104. For example, the warning control signal can indicate that the fluid level is one inch above the outlet 118 of the storage tank 104, the volume of fluid in the storage tank 104 is more than three gallons, the storage tank 104 is full, or the height of the fluid in the storage tank 104 is above five inches. For example, the warning control signal can cause the Ecu 110 to turn off the motor 116.

In some embodiments, in response to determining that the dissolved solids threshold is satisfied, the controller 102 can generate an indicator via a GUI, the indicator indicating that the dissolved solids threshold is satisfied. For example, the controller 102 can generate an indicator via a GUI, the indicator indicating that the dissolved solids threshold is satisfied. The dissolved solids threshold can be satisfied when the indicator indicates that the dissolved solids in the fluid are above or below ten (10) parts per million (PPM). For example, the controller 102 can generate an indicator via a GUI where the indicator can indicate that the dissolved solids threshold is satisfied by displaying the PPM of the fluid in the storage tank 104 on the device 108. For example, the controller 102 can generate an indicator via a GUI, where the indicator can indicate that the dissolved solids threshold is satisfied by causing the GUI to flash blue. For example, the controller 102 can generate an indicator via a GUI, where the indicator indicating that the dissolved solids threshold is satisfied includes an audible alarm. For example, the controller 102 can generate an indicator via a GUI, where the indicator indicating that the dissolved solids threshold is satisfied can change color based on the PPM level, such as green for acceptable levels and red for levels that exceed the threshold. For example, the controller 102 can generate an indicator via a GUI, where the indicator indicating that the dissolved solids threshold is satisfied can include a real-time graph of the dissolved solids levels over time. For example, the controller 102 can generate an indicator via a GUI, where the indicator indicating that the dissolved solids threshold is satisfied can include the indicator triggering a notification to be sent to a remote monitoring system, alerting maintenance personnel of the dissolved solids status. The indicator can activate a secondary system to initiate corrective actions, such as adjusting the filtration process to reduce dissolved solids. For example, the controller 102 can generate an indicator via a GUI, where the indicator indicating that the dissolved solids threshold is satisfied can log the dissolved solids data for future reference and compliance reporting. The controller 102 can generate an indicator via a GUI, where the indicator indicating that the dissolved solids threshold is satisfied can display a visual representation of the dissolved solids distribution within the storage tank 104.

In some embodiments, the controller 102 can generate the indicator to indicate a part per million (PPM) level of the dissolved solids in the fluid. For example, the controller 102 can generate the indicator to indicate a PPM level of the dissolved solids in the fluid indicator where the indication of the PPM of dissolved solids in the fluid displays the PPM level of the dissolved solids in the fluid in real-time. In another example, the controller 102 can generate the indicator to indicate an alert that the PPM level exceeds a predefined threshold and prompt a user of the device 108 to take corrective action. The indicator can log the PPM data for historical analysis and trend monitoring. The indicator can be integrated with a remote monitoring system to provide continuous oversight of the dissolved solids levels. The indicator can include visual cues, such as color changes or flashing lights, to convey the status of the dissolved solids levels to the user.

In some embodiments, the controller 102 can determine that the second fluid level does not satisfy the threshold fluid level. For example, the controller 102 can compare the second fluid level with the threshold fluid level. The controller 102 can determine that the second fluid level satisfies the threshold fluid level by receiving data from the sensors 103 located in the storage tank 104 indicating that the second fluid level of the storage tank 104 is less than one inch above the outlet 118 of the storage tank 104, the volume of fluid in the storage tank 104 is less than three gallons, or the height of the fluid in the storage tank 104 is below five inches, and determining that an amount of fluid in the storage tank satisfies the threshold fluid level. For example, the determination that the second fluid level satisfies the threshold fluid level can include when the second fluid level is above the outlet 118 of the storage tank 104. For example, the controller 102 can utilize additional sensor data to determine the second fluid level does not satisfy the threshold fluid level, such as pressure sensors indicating the fluid pressure within the storage tank 104. For example, the controller 102 can also consider the rate of fluid consumption over time to predict whether the second fluid level will remain above the threshold. For example, if the second fluid level is found to be below the threshold fluid level, the controller 102 can identify that the fluid level is insufficient. The determination can include analyzing trends in fluid consumption to predict potential future deficiencies. In this example, the controller 102 can integrate data from multiple sensors 103 to provide a comprehensive assessment of the fluid level, considering factors such as fluid temperature and pressure.

In some embodiments, the controller 102 can provide a control signal that is configured to cause the pump 112 to operate based on the fluid level satisfying the threshold fluid level. For example, the controller 102 can provide a control signal that is configured to cause the pump 112 to stop operating based on the fluid level satisfying the threshold fluid level. For example, the controller 102 can provide a control signal that is configured to cause the pump 112 to continue operating based on the fluid level satisfying the threshold fluid level. For example, the controller 102 can provide a control signal that is configured to increase or decrease the pump's 112 speed based on the fluid level satisfying the threshold fluid level. For example, the controller 102 can provide a control signal that is configured to increase or decrease the revolutions per minute (RPM) of the pump 112 based on the fluid level satisfying the threshold fluid level. For example, the controller 102 can provide the control signal via the antenna 106. The controller 102 can provide the control signal to the pump 112 via the ECU 110. For example, the ECU 110 can cause the pump 112 to operate based on the fluid level satisfying the threshold fluid level.

In some embodiments, the controller 102 can render the warning via the GUI at a device 108. For example, the warning can cause the GUI to flash, display the warning, or display the fluid level of the storage tank 104. For example, the controller 102 can render the warning via a processor (e.g., processor 304), a graphics processor, or a cloud computer. For example, the controller 102 can render the warning via GUI at another controller 102. For example, the controller 102 can render the warning via the antenna 106. For example, the controller 102 can render the warning to the device 108 to cause the device 108 to generate an output in accordance with the warning.

In some embodiments, the controller 102 can provide the warning control signal to the device 108 to initiate operation of a haptic feedback system of the device 108. The controller 102 can provide the warning control signal to the device 108 via the antenna 106 or via a direct connection to the device 108. In this example, the antenna 106 can provide the warning control signal to the device 108 using the network 114 or via a direct connection to the device 108. For example, the operation of a haptic feedback system of the device 108 can cause the device 108 to vibrate, buzz, or make noise.

In some embodiments, the controller 102 can provide the warning control signal to the device 108 to cause the device 108 to generate an output in accordance with the warning signal. For example, the controller 102 can provide the warning control signal to the device 108 to cause the device 108 to generate a danger output, a flashing output, a sound, or an alarm.

In some embodiments, the controller 102 can render the indicator via a GUI of the device 108. For example, the controller 102 can render the indicator via a GUI of the device 108, the indicator can include the level of dissolved solids in the fluid of the storage tank 104. In another example, the controller 102 can render the indicator via a GUI of the device 108 after the level of dissolved solids has satisfied the threshold level of dissolved solids. For example, the controller 102 can render the indicator via GUI of the device 108, where the indicator causes the GUI of the device 108 to enter different visual states. The controller 102 can render the indicator via the GUI of a device 108 where the indicator causes the GUI of the device 108 to blink, display the PPM of dissolved solids in the fluid, flash multiple times, or change colors.

In some embodiments, the device 108 can provide haptic feedback when receiving a signal. For example, the device 108 can provide haptic feedback when receiving a signal associated with data from the sensors 103 in the storage tank 104. In this example, the device 108 can provide haptic feedback when receiving a signal associated with data from the sensors 103 in the storage tank 104 indicating the storage tank 104 is full of fluid, or the fluid in the storage tank 104 is one inch above the outlet 118. The haptic feedback can include different vibration patterns to indicate different signals being received. For example, a linear vibration pattern can indicate that the fluid level has satisfied the fluid threshold. In this example, the patterned vibration pattern can indicate the number of dissolved solids in the fluid has met the dissolved solids threshold. The signal can include a signal indicating the fluid level has satisfied the threshold fluid level, and the dissolved solids measurement of the fluid has satisfied the threshold dissolved solids measurement of the fluid.

In some embodiments, the controller 102 can provide a sensor signal to a motor 116 indicating low pressure of the fluid, the sensor signal configured to cause the motor 116 coupled to the pump 112 to change operation states. For example, the controller 102 can provide a sensor signal to the motor 116 indicating low pressure of the fluid, the sensor signal configured to cause the motor 116 coupled to the pump 112 to change operation states where the operation states can include operation states configured to increase the motor 116 throttle, decrease the motor 116 throttle, or shut off the motor 116.

In some embodiments, the controller 102 can provide a sensor signal to the motor 116 indicating low pressure of the fluid, the sensor signal configured to cause the motor 116 coupled to the pump 112 to change operation states wherein the sensor signal is indicative of pound per square inch (PSI) of the fluid exiting the pump 112. For example, the sensor signal can indicate that the PSI of the fluid exiting the pump 112 satisfies a threshold pressure level. In this example, when the PSI of the fluid exiting the pump 112 satisfies a threshold pressure level, the controller can provide a sensor signal to the motor 116 where the sensor signal is configured to cause the motor 116 coupled to the pump 112 to change operation states. In some embodiments, the controller 102 can cause the ECU 110 to send a control signal to the pump 112. For example, the controller 102 can cause the ECU 110 to send a control signal to the pump 112 where the control signal includes an indication of low oil pressure in the motor 116.

In some embodiments, the controller 102 can provide the control signal to a fuel control system to adjust a rate of flow of fuel into the motor 116 from a first rate to a second rate, the second rate associated with an idle state of the motor 116. For example, the controller 102 can provide the control signal to the fuel control system to adjust a rate of flow of fuel into the motor 116 from a first rate to the second rate, the second rate associated with a decreased rate of flow of fuel, or an increased rate of flow of fuel. The first rate of flow of fuel into the motor can be associated with a running state of the motor 116, an increased throttle state of the motor 116, or a low throttle state of the motor 116. The fuel control system can include or be a fuel pump, a fuel injection system, a fuel metering unit, the ECU 110, the sensors 103 (e.g., oxygen sensors, fuel pressure sensors, etc.), or actuators. The fuel control system can be coupled to the motor 116. The controller 102 can provide the control signal to a fuel control system to transition a rate of flow of fuel into the motor 116 from a first rate to a second rate, the second rate associated with an idle state of the motor 116.

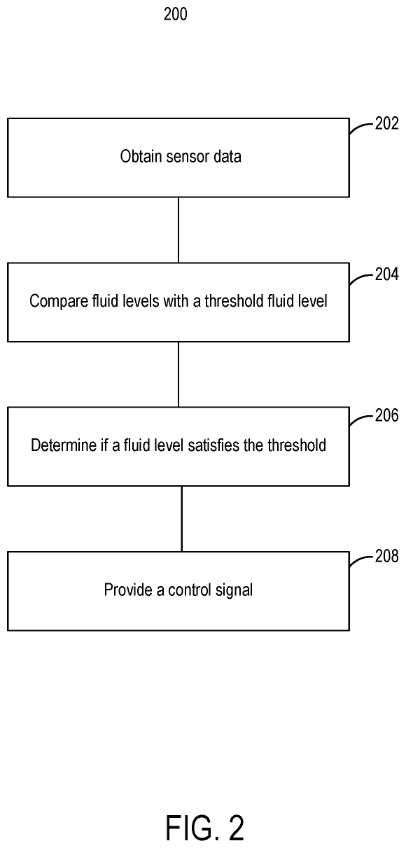

FIG. 2 illustrates a flow diagram of a process for monitoring fluid in a fluid storage tank to optimize fluid availability during operation of the system for system cooling, according to an embodiment.

Referring to FIG. 2, illustrated is a flow diagram of a process 200 for monitoring fluid in a fluid storage tank to optimize fluid availability during operation of the system for system cooling, according to an embodiment. The process 200 includes operations 202-208. However, other embodiments can include additional or alternative operations or can omit one or more operations altogether. The process 200 is described as being executed by a controller, which can be the same as, or similar to, the controller 102 described in FIG. 1.

At operation 202, the controller can obtain sensor (e.g., that is the same as, or similar to, the sensor 103 of FIG. 1) data can obtain sensor data associated with a fluid level measured in a storage tank (e.g., that is the same as, or similar to, the storage tank 104 of FIG. 1). For example, the controller can obtain sensor data associated with a fluid level, where the sensor data is indicative of the fluid level, a temperature of the fluid, or an amount of fluid in the storage tank similar to as described above in system 100 of FIG. 1. For example, the controller 102 can obtain sensor data from at least one sensor located in or on the storage tank. There can be multiple sensors in or on the storage tank that measure the fluid level in the storage tank. The sensors can include ultrasonic sensors, photoelectric sensors, float switches, temperature sensors, capacitive sensors, conductive sensors, magneto strictive sensors, and radar sensors.

At operation 204, the controller can compare fluid levels measured in the storage tank with a threshold fluid level representing a minimum level at which a pump (e.g., that is the same as, or similar to, the pump 112 of FIG. 1) can operate safely. For example, the controller can compare the fluid level measured in the storage tank with a threshold fluid level representing a minimum level at which a pump can operate. For example, the controller can compare the fluid level measured in the storage tank with a threshold fluid level representing a maximum fluid capacity of the storage tank. For example, the controller can compare the fluid level measured in the storage tank with a threshold fluid level representing a minimum level at which a pump can operate. The threshold fluid level representing a minimum level at which a pump can operate safely can include at least one inch of fluid above the outlet of the storage tank, five gallons of fluid, ten percent of storage tank capacity, or ten inches of fluid. For example, the fluid level can include the fluid in the storage tank. For example, the pump can operate safely when there is at least two gallons of water present in the storage tank. For example, the pump can operate safely when there is enough fluid in the storage tank to fully engage the pump. For example, the pump can operate safely when there is sufficient fluid to cool the pump. For example, the pump can operate safely when the pump is functioning at its maximum volumetric capacity.

At operation 206 in response to comparing the fluid level with the threshold fluid level, the controller can determine that the fluid level satisfies the threshold fluid level. For example, the controller can determine that the fluid level satisfies the threshold fluid level by receiving data from the sensors located in the storage tank indicating that the fluid level of the storage tank is less than one inch above the outlet (e.g., that is the same as, or similar to, the outlet 118 of FIG. 1) of the storage tank, the volume of fluid in the storage tank is less than three gallons, or the height of the fluid in the storage tank is below five inches, and determining that an amount of fluid in the storage tank satisfies the threshold fluid level. For example, the determination that the fluid level satisfies the threshold fluid level can be included when the fluid level is above the outlet of the storage tank. For example, the controller can integrate data from multiple sensors to provide a more accurate assessment of the fluid level, considering factors such as fluid temperature and pressure.

At operation 208, the controller can provide a control signal that is configured to cause the pump to operate based on the fluid level satisfying the threshold fluid level. For example, the controller can provide a control signal that is configured to cause the pump to stop operating based on the fluid level satisfying the threshold fluid level. For example, the controller can provide a control signal that is configured to cause the pump to continue operating based on the fluid level satisfying the threshold fluid level. For example, the controller can provide a control signal that is configured to increase or decrease the pump's speed based on the fluid level satisfying the threshold fluid level. For example, the controller can provide a control signal that is configured to increase or decrease the revolutions per minute (RPM) of the pump. For example, the controller can provide the control signal via the antenna (e.g., that is the same as, or similar to, the antenna 106 of FIG. 1). The controller can provide the control signal to the pump via the ECU. For example, the ECU (e.g., that is the same as, or similar to, the ECU 110 of FIG. 1) can cause the pump to operate based on the fluid level satisfying the threshold fluid level.

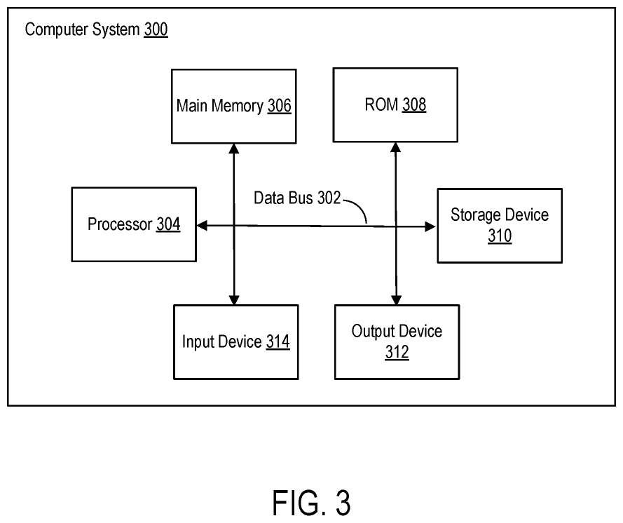

FIG. 3 illustrates a block diagram of an example computer system 300 also referred to as a computer system 300. The computer system 300 can include, included by, or be used to implement the system 100, or any components of the system 100, such as the controller 102, the device 108, or the ECU 110. The computer system 300 includes at least one data bus 302 or other communication component for communicating information and at least one processor 304 or processing circuit coupled to the data bus 302 for processing information. The computer system 300 can also include one or more processors 304 or processing circuits coupled to the data bus 302 for processing information. The computer system 300 also includes at least one main memory 306, such as a random-access memory (RAM) or other dynamic storage device, coupled to the data bus 302 for storing information, and instructions to be executed by the processor 304. The main memory 306 can be used for storing information during execution of instructions by the processor 304. The computer system 300 may further include at least one read only memory (ROM) 308 or another static storage device coupled to the data bus 302 for storing static information and instructions for the processor 304. A storage device 310, such as a solid-state device, magnetic disk or optical disk, can be coupled to the data bus 302 to persistently store information and instructions.

The computer system 300 may be coupled via the data bus 302 to an output device 312, such as a liquid crystal display, or active-matrix display, for displaying information to a user. An input device 314, such as a keyboard or voice interface may be coupled to the data bus 302 for communicating information and commands to the processor 304. The input device 314 can include a touch screen display. The input device 314 can also include a cursor control, such as a mouse, a trackball, or cursor direction keys, for communicating direction information and command selections to the processor 304 and for controlling cursor movement on the output device 312, such as a display.

The processes, systems and methods described herein can be implemented by the computer system 300 in response to the processor 304 executing an arrangement of instructions contained in main memory 306. Such instructions can be read into main memory 306 from another computer-readable medium, such as the storage device 310. Execution of the arrangement of instructions contained in main memory 306 causes the computer system 300 to perform the illustrative processes described herein. One or more processors in a multi-processing arrangement may also be employed to execute the instructions contained in main memory 306. Hard-wired circuitry can be used in place of or in combination with software instructions together with the systems and methods described herein. Systems and methods described herein are not limited to any specific combination of hardware circuitry and software.

Although an example computing system has been described in FIG. 3, the subject matter including the operations described in this specification can be implemented in other types of digital electronic circuitry, or in computer software, firmware, or hardware, including the structures disclosed in this specification and their structural equivalents, or in combinations of one or more of them.

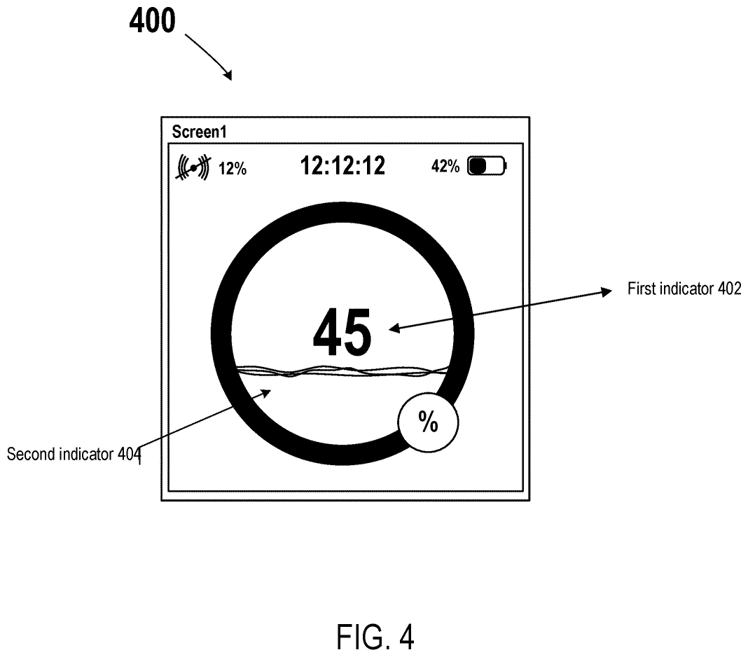

FIG. 4 illustrates an example implementation of a user interface (UI) displayed on the device 108, according to an embodiment. As illustrated, the device 108 can be configured to output a user interface thereon. The user interface can include a display of the fluid level of the storage tank 104 on a GUI 400. In some embodiments, the device 108 can generate data that causes a graphical user interface to be displayed on a device as described here in. For example, the device 108 can generate GUI data that is configured to cause the display device of a device 108 to generate a display indicating one or more features described herein. For example, the GUI 400 can include a first indicator 402, the first indicator 402 designed to display an amount of water in the storage tank 104 using a percentage. The GUI 400 can include a second indicator 404, the second indicator 404 designed to display an amount of water in the storage tank 104 using a virtual display of water.

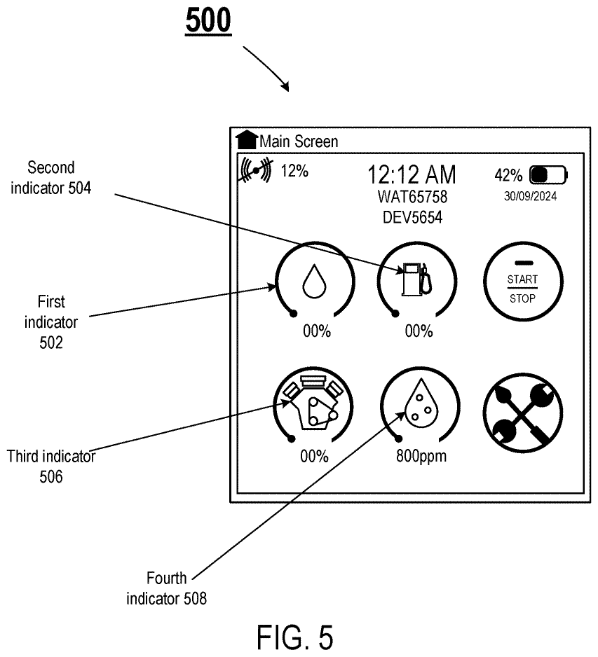

FIG. 5 illustrates an example implementation of a user interface (UI) displayed on the device 108, according to an embodiment. As illustrated, the device 108 can be configured to output a user interface thereon. The user interface can include a display of the fluid level of the storage tank 104, the fuel level of the motor 116, a virtual button configured to cause the device 108 to send a signal to the motor 116, the signal associated with a stop or start instruction, the throttle level of the motor 116, or the dissolved solids level of the fluid in the storage tank 104 on a GUI 500. In some embodiments, the device 108 can generate data that causes a graphical user interface to be displayed on a device as described here in. For example, the device 108 can generate GUI data that is configured to cause the display device of the device 108 to generate a display indicating one or more features described herein. For example, the GUI 500 can include a first indicator, the first indicator designed to display an amount of water in the storage tank 104 using a percentage amount. The GUI 500 can include a first indicator 502, the first indicator 502 designed to display an amount of water in the storage tank 104 using a percentage amount. The GUI 500 can include a second indicator 504, the second indicator 504 designed to display an amount of fuel in the motor 116 using a percentage amount. The GUI 500 can include a third indicator 506, the third indicator 506 designed to display the load percentage of the motor 116 using a percentage amount. The GUI 500 can include a fourth indicator 508, the fourth indicator 508 designed to display the dissolved solids percentage of fluid in the storage tank 104 using a percentage amount.

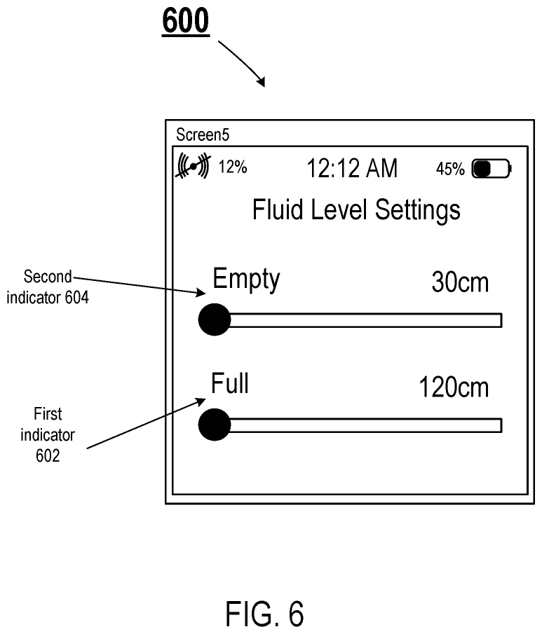

FIG. 6 illustrates an example implementation of a user interface (UI) displayed on the device 108, according to an embodiment. As illustrated, the device 108, can be configured to output a user interface thereon. The user interface can include a display of a fluid level setting on a GUI 600. In some embodiments, the device 108 can generate data that causes a graphical user interface to be displayed on a device as described here in. For example, the device 108 can generate GUI data that is configured to cause the display device of the device 108 to generate a display indicating one or more features described herein. For example, the GUI 600 can include a first indicator 602, the first indicator 602 designed to display a maximum amount of fluid the storage tank 104 will hold before the sensor 103 will indicate to the controller 102 that the storage tank 104 is full. For example, the GUI 600 can include a second indicator 604, the first indicator designed to display a maximum amount of fluid the storage tank 104 will hold before the sensor 103 will indicate to the controller 102 that the storage tank 104 is full.

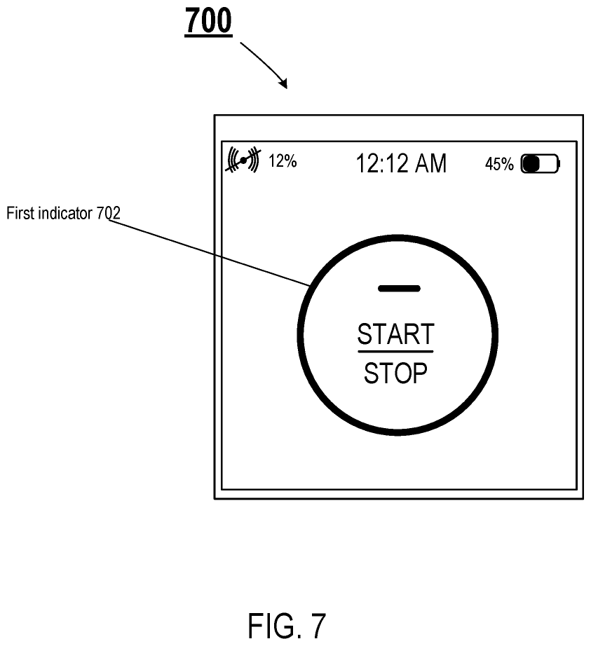

FIG. 7 illustrates an example implementation of a user interface (UI) displayed on the device 108, according to an embodiment. As illustrated, the device 108 can be configured to output a user interface thereon. The user interface can include a display of a virtual button on a GUI 700. The virtual button can be configured to cause the device 108 to send a signal to the motor 116, the signal associated with a stop or start instruction. In some embodiments, the device 108 can generate data that causes a graphical user interface to be displayed on a device as described herein. For example, the device 108 can generate GUI data that is configured to cause the display device of the device 108 to generate a display indicating one or more features described herein. For example, the GUI 700 can include a first indicator 702, the first indicator designed to display an interactive virtual button that can send a stop or start signal to the motor 116 via the antenna 106.

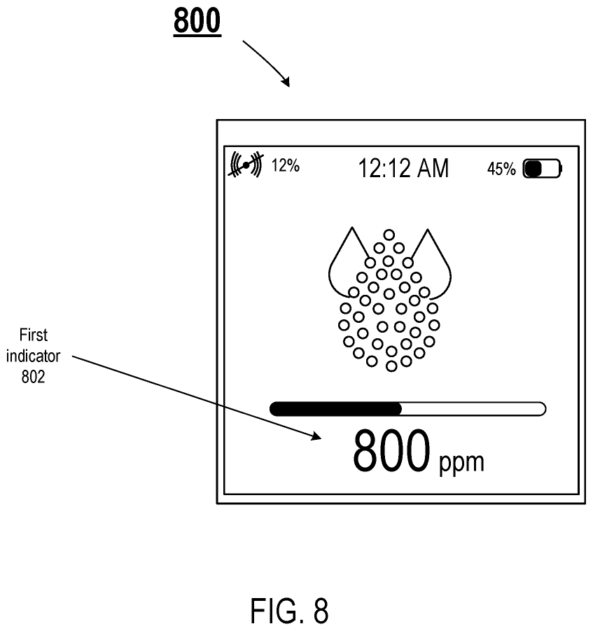

FIG. 8 illustrates an example implementation of a user interface (UI) displayed on the device 108, according to an embodiment. As illustrated, the device 108 can be configured to output a user interface thereon. The user interface can include a display of a measurement of the PPM of the dissolved solids in fluid, the measurement associated with the PPM of dissolved solids in fluid of the storage tank 104 on a GUI 800. In some embodiments, the device 108 can generate data that causes a graphical user interface to be displayed on a device as described herein. For example, the device 108 can generate GUI data that is configured to cause the display device of the device 108 to generate a display indicating one or more features described herein. For example, the GUI 800 can include a first indicator 802, the first indicator 802 designed to display an interactive virtual button that can send a stop or start signal to the motor 116 via the antenna 106.

FIG. 9 illustrates an example implementation of a PCB 900, according to an embodiment. As illustrated, the PCB 900 can be or include the controller 102 or any controller as described herein. The PCB 900 can include an antenna 902, a processor 904, main memory 906, a ROM 908. The PCB 900 can be mechanically coupled to the controller 102. The PCB 900 can be or include a transmitter. The PCB 900 can be connected to the storage tank 104, the device 108, the pump 112, or the motor 116 wirelessly or with a wired connection. The PCB 900 can be or include the antenna 106 or any antenna as described herein. The PCB 900 can be or include the processor 304 or any processor as described herein. The PCB 900 can be or include main memory 306 or any memory as described herein. The PCB 900 can be or include ECU 110 or any memory as described herein.

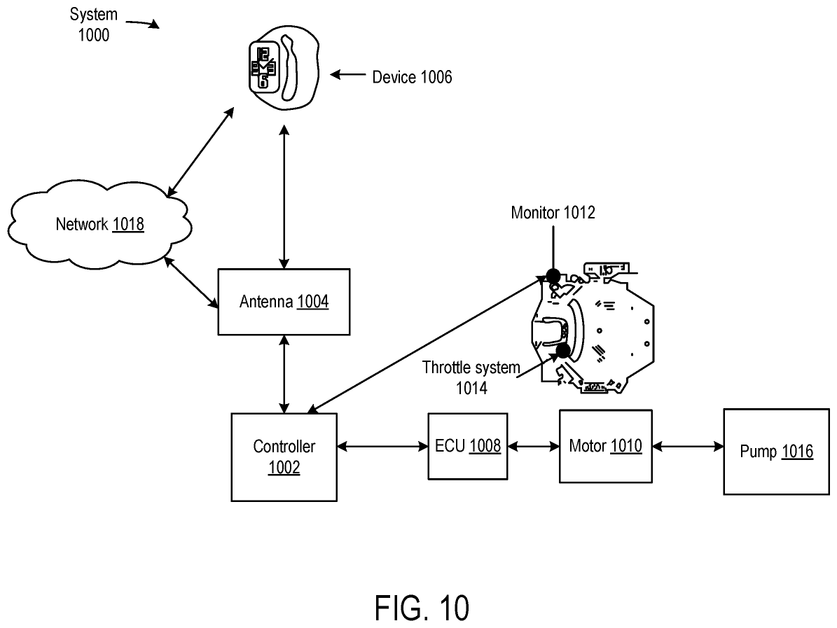

FIG. 10 shows components of a system 1000 for controlling a motor by dynamically adjusting operation of an energy delivery system to maintain a set number of rotations per minute in accordance with a set point, in accordance with one or more embodiments. For example, the system 1000 can include a controller 1002, an antenna 1004, a device 1006, an ECU 1008, a pump 1016, a network 1018, a motor 1010, a monitor 1012, and a throttle system 1014. The controller 1002, the antenna 1004, the device 1006, the ECU 1008, the pump 1016, the network 1018, the motor 1010, the monitor 1012, and the throttle system 1014 can be configured to interconnect using one or more wired and/or wireless connections. Various components depicted in FIG. 10 can be included in a pressure washer system, which individuals operate to clean various objects (e.g., structures, buildings, etc.) that includes one or more pressure washer pumps (e.g., the pump 1016). While the system 1000 is shown as including various components, it will be understood that similar systems are not confined to the components described herein and can include additional or other components, that are the same as, or similar to, those described. For example, one or more components of the system 1000 of FIG. 10 can be the same as, or similar to, one or more components of the system 100 of FIG. 1 that are similarly-named or similarly-described. Some or all of the components discussed with respect to system 1000 can be connected to each other through a network 1018 using one or more wired or wireless communication connections, which can be the same or similar to the network 114.

In some embodiments, the controller 1002 can include a device that is the same, or similar to, the controller 102 in FIG. 1. For example, the controller 1002 can include any appropriate logic device that can be adapted to receive, store, and/or execute appropriate instructions generated based on operation of the device 1006. In examples, the controller 1002 can be configured to execute instructions to implement one or more operations when controlling operation of the motor 1010, such as obtaining monitor data associated with the state of the motor 1010 (e.g., speed of a motor, position of a servo motor) or the state of the throttle system 1014 (e.g., position of a throttle plate, angle of a valve, position of a servo motor). In some embodiments, the controller 1002 can be configured to then process the monitor data, provide control signals (e.g., through the controller 1002) to the ECU 1008, generate indicators of the RPMs at which the motor 1010 is set, etc. In an example, the controller 1002 can be configured to periodically or continuously compare a speed of the motor 1010 to a hold speed established by user input provided at the device 1006 as well as information regarding the state of the motor 1010 (e.g., the RPMs at which the motor is operating) and iteratively update the operation of the motor 1010 to cause the motor 1010 to increase/decrease RPMs to maintain operation of the motor 1010 at a set RPM value (or range of values). While discussed as an independent computing device, the controller 1002 can be partially and/or completely implemented by one or more other devices of FIG. 10, such as by the ECU 1008 of FIG. 10. In some embodiments, the controller 1002 can be configured to be in direct or indirect communication with the antenna 1004, the device 1006, the ECU 1008, the pump 1016, the network 1018, the motor 1010, the monitor 1012, and the throttle system 1014.

In some embodiments, the device 1006 can include a device that is the same, or similar to, the device 108 and can include any computing device capable of performing the various tasks and processes described herein. For example, the controller 1002 can be configured to generate an indicator to indicate a rotations per minute (RPM) value. The RPM value can be associated with a hold speed for operation of the system 1000, set by a user controlling a device (e.g., the same, or similar to the device 1006). For example, the controller 1002 can cause a display device of the device 1006 to generate an output that renders the indicator via a graphical user interface (GUI) thereon. In operation, various users, such as individuals operating the system 1000, can access the GUI managed by the device 1006 to monitor the speed of the motor 1010 and the configuration of the throttle system 1014. In some embodiments, various users can input a hold speed for the motor 1010 through the device 1006 for example by using a GUI, a knob, or entering a numeric value associated with an RPM.

In some embodiments, the ECU 1008 can include a device that is the same, or similar to, the ECU 110 and can include any computing device, comprising a processor and a non-transitory machine-readable storage medium capable of performing the various tasks and processes described herein. In some embodiments, the ECU 1008 can be managed by the controller 1002 to control the motor 1010, the monitor 1012, and the throttle system 1014.

In some embodiments, the motor 1010 can include a device that is the same as, or similar to, the motor 116 and can include a device or system configured to power or cause movement of various components or systems as described herein. In some examples, the motor 1010 can include one or more monitors 1012. For example, the ECU 1008 can manage the motor 1010 by sending signals to and/or receiving signals from the motor 1010. In examples, the motor 1010 can include the throttle system 1014 that can adjust the speed of the motor 1010. In some embodiments, the motor 1010 can be associated with a model. The model can include a manufacturer and model number, manufacturer part number, serial number, or other identifier of the model.

In some embodiments, the monitor 1012 can be any type of monitor used for providing information associated with the motor 1010. For example, the monitor 1012 can provide information about the speed or position (e.g., an angle of a servo motor) of the motor 1010. In examples, the monitor 1012 can include inductance wire sensors, other hall effect sensors, tachometers, encoders, or proximity sensors that can provide information about the speed of the motor 1010 in rotations per minute (RPMs). The one or more monitors 1012 can be placed along, on, inside, or near the any component of the motor 1010. In an embodiment, an inductance wire sensor located on the spark plug wire of the motor 1010 can be used to provide information about the speed of the motor 1010 in RPMs.

In some embodiments, the throttle system 1014 can include any device or system configured to adjust the speed of the motor 1010 as described herein. For example, where the motor 1010 includes an internal combustion motor, the throttle system 1014 can include a throttle body, a throttle plate, a valve, a throttle position sensor (TPS), an electronic throttle control motor (ETC), a servo motor, an automatic throttle control, a choke, an encoder, etc. In this example, various components of the throttle system can cooperate to adjust the amount of air fuel mixture entering the motor 1010, which in turn adjusts the speed of the motor 1010. In some embodiments, the throttle system 1014 can send data associated with the configuration and state of components of the throttle system 1014 to the ECU 1008 and/or the controller 1002. For example, a throttle position sensor in the throttle system 1014, can send data associated with the position of the throttle plate to the ECU 1008 and/or the controller 1002. In some embodiments, the ECU 1008 can manage the throttle system 1014 by sending signals to the throttle system 1014. In examples, the ECU 1008 and/or the controller 1002 can send a signal to a servo motor in the throttle system 1014 that can change the position of the servo motor, which can adjust the throttle plate to increase or decrease the amount of air entering the throttle system 1014. In some embodiments, component of the throttle system 1014 can operate at a plurality of positions and settings. For example, a servo motor in the throttle system 1014 can operate at a plurality of angles, including a widest angle, proportionally wider or narrower angles, and narrowest angle, and the encoder of the servo motor can send data associated with the angle of the servo motor to the ECU 1008 and/or the controller 1002. In embodiments, a valve in the throttle system 1014 can operate at a plurality of angles as described above, including the widest angle and the narrowest angle.

In some embodiments, the pump 1016 can include a device that is the same, or similar to, the pump 112 and can include any components configured to, alone or in cooperation, move fluids, gases, or other flowable materials through a conduit or system as described herein. For example, the ECU 1008 can manage the pump 1016 by sending signals to the pump 1016. In some examples, the motor 1010 can change the fluid flow through the pump 1016 by the motor 1010 increasing or decreasing speed of a component (e.g., of a rotor, etc.) coupled to the pump 1016 to correspondingly increase or decrease the rate at which the pump 1016 operates to increase or decrease fluid flow therethrough.

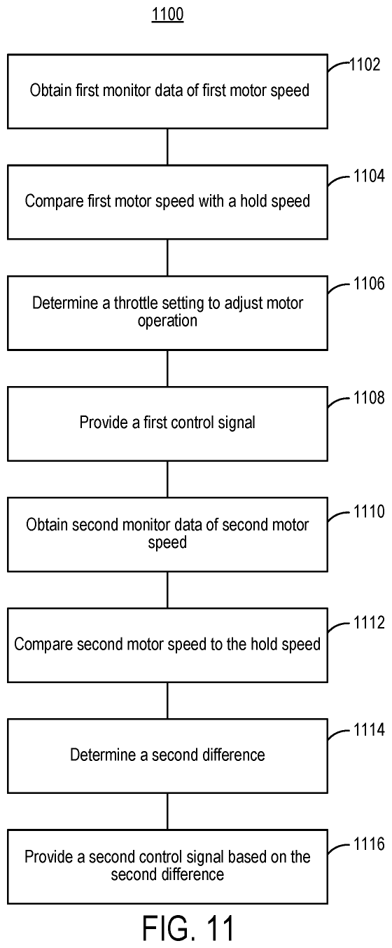

Referring to FIG. 11, illustrated is a flow diagram of a process 1100 for controlling a motor by dynamically adjusting operation of an energy delivery system to maintain a set number of RPM in accordance with a set point. The process 1100 includes operations 1002-1016. However, some embodiments can include additional or alternative operations or can omit one or more operations altogether. While certain aspects of the process 1100 are described as being performed by a controller (e.g., that is the same as, or similar to, the controller 102 of FIG. 1 and/or the controller 1002 of FIG. 10), it will be understood that any suitable combination of components from a system that is the same as or similar to the system 100 of FIG. 1 and/or the system 1000 of FIG. 10 (or one or more components thereof) can be configured to implement the process 1100.

At operation 1102, the process 1100 can include obtaining monitor data (e.g., first monitor data) associated with a motor speed (e.g., a first motor speed) of a motor. For example, a controller can be configured to obtain the monitor data based on operation of a motor (e.g., that is the same as, or similar to, the motor 1010 of FIG. 10). In some embodiments, the controller can obtain the monitor data associated with a first motor speed from a monitor (e.g., that is the same as, or similar to, the monitor 1012), where the monitor data is indictive of a value representing a speed of the motor measured in RPMs. In examples where the monitor data is not directly correlated with the speed of the motor, the controller can convert the monitor data into a value that represents the speed of the motor. This can include, for example, converting a number of times a spark plug of the motor generates spark per second into a value representing the RPMs of the motor. In some examples, the monitor can include one or more components as described herein, such as an inductance wire sensor on a spark plug of the motor, a hall effect sensor, a tachometer, an encoder, or a proximity sensor.

At operation 1104, the process 1100 can include comparing the first motor speed with a hold speed. For example, the controller can determine the hold speed based on input received by a user at a device (e.g., that is the same as, or similar to, the device 1006 of FIG. 10). The hold speed can be a set speed in RPMs at which the user is configuring the motor to operate at. The controller can be configured to compare the first motor speed with a hold speed in response to the user device receiving input of a hold speed.

In some embodiments, the controller can then determine a difference (e.g., a first difference) between the first motor speed and the hold speed. In this example, the controller can receive data that is associated with (e.g., represents) the hold speed from the device based on the user interacting with an input device (e.g., a dial, knob, touchscreen, etc.) of the device to set an RPM value. The controller can then calculate the relative difference between the hold speed and the first motor speed to determine the first difference. In embodiments, the controller can calculate the first difference based on an absolute difference, relative difference, percent difference, squared difference, ratio, derivative, mean, or other measures.

In some embodiments, the controller can also be configured to generate an indicator to indicate an RPM value associated with the hold speed via a graphical user interface (GUI) on the user device. For example, the controller can determine a default hold speed (e.g., that is hardcoded or set by a user during initial configuration of the system 1000). The controller can then render the indicator using a display device of the user device. For example, the indicator can include an RPM value, a dial, gauge, point on a line graph, or other visual representation. In examples, the display device can include a touch screen, organic light emitting diode (OLED) screen, liquid crystal display (LCD), or any other suitable display device.

At operation 1106, the process 1100 can include determining a throttle setting (e.g., a first throttle setting) based on the first difference to adjust operation of the motor and/or a throttle system (e.g., the same or similar to the throttle system 1014) and reduce the first difference. For example, the controller can determine the first throttle setting based on the first difference and configure the throttle system to adjust energy delivered to the motor to adjust (e.g., increase and/or decrease) a speed of the motor to reduce the first difference. In some embodiments, the throttle setting can include an amount to open or close a component of the throttle system to for a set period of time, a set angle to position a component of the throttle system at for a set period of time, or a series of operations to perform on the throttle system. In some examples, the controller and/or the ECU (e.g., that is the same as, or similar to, the ECU 1008) can determine the throttle setting based on comparing the first difference to one of more threshold differences to select and threshold difference. And in these examples, each threshold difference can be associated with a predetermined increase or decrease of energy to the motor to determine a throttle setting.

In some embodiments, the controller can be further configured to receive user input data associated with a plurality of user inputs indicating positions of the throttle system to determine a set of calibration throttle settings. For example, these positions can include a specific angle of a servo motor in the throttle system, a position of the throttle plate, a position of a valve in the throttle system, etc. In examples, a first position in the plurality of user inputs indicating positions can correspond to a position where the throttle system is at its widest angle, and a second position in the plurality of user inputs indicating positions can correspond to a position where the throttle system is at its narrowest angle. In some embodiments, in response to receiving the plurality of user inputs indicating the positions of the throttle system, the controller can be configured to obtain a plurality of motor sensor signals associated with a plurality of sensed positions of the throttle system. For example, the controller and/or the ECU can receive the sensed position of the throttle plate, the sensed position or angle of the servo motor from the encoder of the servo motor, or a sensed position of a valve in the throttle system. In examples, the controller can configure the set of calibration throttle settings based on the plurality of user inputs indicating the positions of the throttle system and the plurality of sensed positions of the throttle system. For example, the controller can perform a linear regression, polynomial regression, error minimization technique, etc., on the plurality of user inputs indicating the positions of the throttle system, and the plurality of sensed positions of the throttle system to configure the set of calibration throttle settings. In some embodiments, the controller can calibrate the first throttle setting, based on the first difference to adjust operation of the motor, to reduce the first difference with the set of calibration throttle settings. In examples, the calibration throttle settings can include a lookup table between the first difference to a change in position of the servo motor, a range of positions, or a linear regression between the first difference and the range of positions in the calibration settings.

In some embodiments, the controller can be configured to determine that a motor (e.g., a first motor) was replaced and reconfigure one or more throttle settings. For example, the controller can determine that the motor that was replaced was used to calibrate the throttle settings implemented by the controller. The controller can then compare a type of the motor that was replaced with a type of the new motor and determine whether the two are compatible. For example, in response to obtaining an indication that the first motor was replaced with a different motor (e.g., a second motor), the controller can be configured to determine that at least one previous set of calibration throttle settings for the first model are or are not associated with the second motor. In doing so, the controller can compare a model type associated with a new motor (e.g., a motor configured for updated operation) and a model type associated with a replaced motor (e.g., a previous motor configuration). The controller can then determine whether the model types are compatible (e.g., when the model types match or share similar configurations) and can populate a lookup table or a database (e.g., an information storage repository) associated with the replaced motor with previous calibration throttle settings. In some examples, the controller can be configured to save calibration throttle settings in the lookup table or database and can be configured to recall the stored calibration throttle settings from the lookup table or database. In another example, if the compatibility criteria are satisfied, the controller can be configured to retrieve a previous set of calibration throttle settings associated with the replaced motor (e.g., a first model) and can configure the new motor with the retrieved calibration throttle settings.

At operation 1108, the process 1100 can include providing a control signal (e.g., a first control signal) to a throttle system that can be configured to adjust operation of the throttle system based on the throttle setting. For example, the controller can provide a control signal that is configured to change the speed of the motor. In some examples, the controller can send a first control signal that can cause the ECU to adjust the throttle of the motor based on the throttle settings. In examples, the controller can send a control signal that is configured to cause the motor to operate at the hold speed for a period a time. In embodiments, the controller can send a control signal that is configured to increase or decrease the speed of the motor to reduce the difference between the motor speed and the hold speed for a period of time. It will be understood that the controller can provide the control signal via an antenna (e.g., the same or similar to the antenna 1004).

At operation 1110, the process 1100 can include obtaining monitor data associated with a speed of the motor (e.g., a second speed of the motor) at a point in time after the first speed was obtained. In some embodiments, the controller can obtain second monitor data (e.g., the same or similar to the first monitor data) associated with the second motor speed from a monitor (e.g., the same or similar to the monitor 1012). The second monitor data can have properties the same or similar to the first monitor data. In response to the controller obtaining monitor data associated with the first speed or the second speed of the motor, the controller can be configured to generate an indicator to indicate an RPM. The RPM value can be associated with the first speed or the second speed of the motor. In some examples, the controller can render the indicator via a graphical user interface (GUI) using a display device of the user device. For example, the controller can generate the GUI that includes the indicator displayed thereon. In this example, the controller can generate and provide GUI data associated with (representing) the GUI, the GUI data configured to cause the display device of the user device to generate and display the GUI thereon.

At operation 1112, the process 1100 can include comparing the second speed of the motor to the hold speed. In some embodiments, the controller 1002 can perform a comparison between the second speed of the motor and the hold speed. In examples, this comparison can be the same as, or similar to, the comparison performed by the controller between the first speed of the motor and the hold speed. At operation 1114, the process 1100 can include determining a difference (e.g., a second difference) between the second speed of the motor and the hold speed. For example, the controller can compute the second difference in a manner similar to the comparison of the hold speed to the first difference. The controller can then determine one or more adjustments to be made to the operation of the motor based on the second difference. For example, where the second difference satisfies a threshold value (e.g., indicating that the second difference is greater than a predetermined difference), the controller can generate a control signal to adjust operation to reduce the second difference. In instances where the controller generates a control signal to adjust operation, where the second speed of the motor is greater than the hold speed, the controller can generate the control signal to cause the motor to reduce speed (e.g., by a predetermined amount, etc.). Alternatively, in instances where the controller generates a control signal to adjust operation, where the second speed of the motor is greater than the hold speed, the controller can generate the control signal to cause the motor to reduce speed (e.g., by a predetermined amount, etc.).

At operation 1116, the process 1100 can include providing a control signal (e.g., a second control signal) to adjust operation of the throttle system based on the second difference. The second control signal can be the same or similar to the first control signal. For example, as described above, the controller can configure the second control signal to change the speed of the motor based on the second difference. In embodiments, the controller can determine the second control signal based on the second difference and additional information, such as the time since the first control signal was provided, a comparison between the first speed of the motor and the second speed of the motor, and the current configuration of the throttle system. In embodiments, the second control signal can include a second throttle setting, the same, or similar to, the first throttle setting. In examples, the controller and/or the ECU can determine a second control signal using information in addition to the second difference, such as information associated with the timing of obtaining the first difference and second difference, the magnitude of the difference, the sign of the difference, a current throttle position, or a configured range of the throttle system.

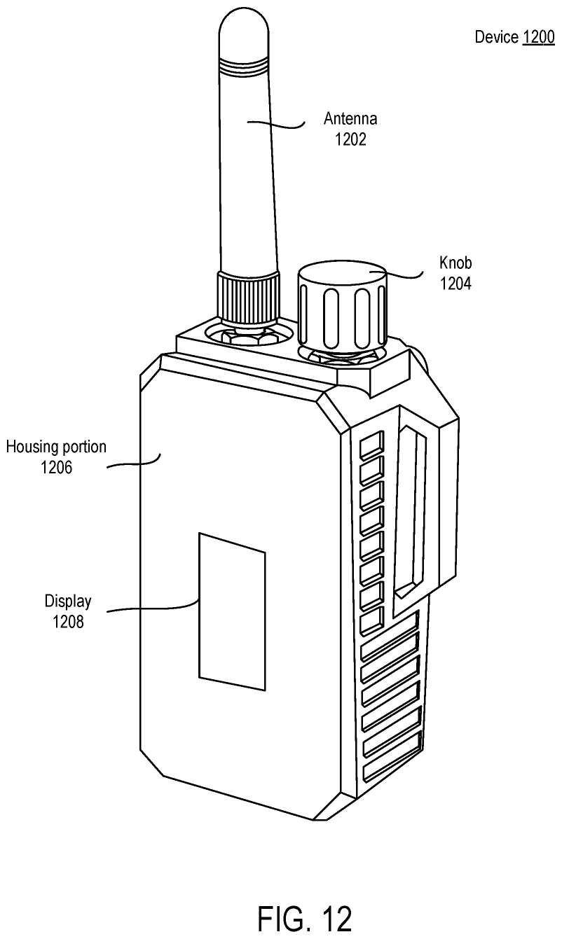

FIG. 12 illustrates an example implementation of a user device, a device 1200, according to an embodiment. The device 1200 can include a device that is the same, or similar to, the device 1006. For example, the device 1200 can include an antenna 1202, a knob 1204, a housing portion 1206, and a display 1208. The device 1200 can be connected to the controller 1002. The device 1200 can include the knob 1204, which can be adjusted by a user, to set a hold speed, and the device 1200 can send data indicating this hold speed to the controller 1002 via the antenna 1202. For example, the antenna 1202 can be the same or similar to the antenna 1004. In some embodiments, the device 1200 can include the housing portion 1206, on at least one face of the device 1200, which can house the display 1208. In some embodiments, the device 1200 can generate GUI data configured to cause the device 1200 to generate a visualization indicating one or more features on the display 1208. For example, the display 1208 can present a GUI interface, an indicator, a dial, or a visual representation of a hold speed or a measured speed of a motor (e.g., the same, or similar to, the motor 1010). In some embodiments, the display 1208 can include a touch screen to receive user input data. In some embodiments, the user input data received on the display 1208 can configure the device 1200 to enter a mode for calibration, where the device 1200 can receive a plurality of user inputs indicating positions of a throttle system (e.g., the same or similar to the throttle system 1014) to determine a set of calibration throttle settings. For example, a user can input data indicating the positions of a throttle system through a GUI on the display 1208 and/or through inputs configured from the knob 1204.

While various aspects and embodiments have been disclosed, other aspects and embodiments are contemplated. The various aspects and embodiments disclosed are for purposes of illustration and are not intended to be limiting, with the true scope and spirit being indicated by the following claims.

The present techniques will be better understood with reference to the following enumerated embodiments:
1. Methods for monitoring fluid in storage tanks to operate a pump.
2. The method of any one of the preceding embodiments, further comprising: obtaining sensor data associated with a fluid level measured in a storage tank; comparing the fluid level measured in the storage tank with a threshold fluid level representing a minimum level at which a pump can operate safely; in response to comparing the fluid level with the threshold fluid level, determining, by one or more processors, that the fluid level satisfies the threshold fluid level; and providing, by the one or more processors, a control signal that is configured to cause the pump to operate based on the fluid level satisfying the threshold fluid level.
3. The method of any one of the preceding embodiments, further comprising: in response to determining that the fluid level satisfies the threshold fluid level a warning via a GUI, the warning indicating that the fluid level in the storage tank is below a safe operation level; and rendering the warning via a GUI of a device.
4. The method of any one of the preceding embodiments, further comprising: in response to determining that the fluid level satisfies the threshold fluid level, generating a warning control signal; and providing, by one or more processors, the warning control signal to a device to initiate operation of a haptic feedback system of the device.
5. The method of any one of the preceding embodiments, wherein the sensor data comprises first sensor data, the method further comprising: obtaining second sensor data associated with a measurement of dissolved solids in fluid stored in the storage tank; comparing, by one or more processors, the measurement of the dissolved solids with a dissolved solids threshold to determine that the dissolved solids threshold is satisfied; in response to determining that the dissolved solids threshold is satisfied, generating, by one or more processors, an indicator via a GUI indicating that the dissolved solids threshold is satisfied; and rendering the indicator via the GUI of a device.
6. The method of any one of the preceding embodiments, wherein generating the GUI comprises: generating the GUI to indicate a part per million (PPM) level of the dissolved solids in the fluid; wherein the one or more processors configured to cause the GUI to be displayed are configured to: providing, by the one or more processors, GUI data associated with the GUI to a device that is configured to receive the GUI data and generate a user interface in accordance with the GUI.
7. The method of any one of the preceding embodiments, wherein providing a control signal comprises: providing a sensor signal to a motor indicating low pressure of fluid, the sensor signal configured to cause the motor coupled to the pump to change operation states.
8. The method of any one of the preceding embodiments, wherein providing a control signal comprises: providing the control signal to a fuel control system to adjust a rate of flow of fuel into a motor to transition from a first rate to a second rate, the second rate associated with an idle state of the motor.
9. The method of any one of the preceding embodiments, wherein the sensor data comprises first sensor data, the method further comprising: obtaining second sensor data associated with a second fluid level measured in the storage tank at a point in time after the first sensor data is generated; determining, by the one or more processors, that the second fluid level does not satisfy the threshold fluid level; and providing, by the one or more processors, a second control signal that is configured to cause the fuel control system to adjust the rate of flow of fuel into the motor to transition from the second rate to the first rate.
10. A method for controlling a motor by dynamically adjusting operation of an energy delivery system to maintain a set number of rotations per minute in accordance with a set point.
11. The method of any one of the preceding embodiments, further comprising: in response to obtaining first monitor data associated with a first speed of a motor, comparing, by one or more processors, the first speed to a hold speed to determine a first difference, the hold speed being set based on input received by a user; determining, by the one or more processors, a throttle setting based on the first difference to adjust operation of the motor and reduce the first difference; providing, by the one or more processors, a first control signal to a throttle system to adjust operation of the throttle system based on the throttle setting; in response to providing the first control signal, obtaining, by the one or more processors, second monitor data associated with a second speed of the motor; comparing, by the one or more processors, the second speed to the hold speed to determine a second difference; and providing, by the one or more processors, a second control signal to adjust operation of the throttle system based on the second difference.
12. The method of any one of the preceding embodiments, further comprising: obtaining, by the one or more processors, input data associated with an input received at a user device indicating the hold speed, wherein comparing the first speed of the motor to the hold speed comprises: comparing, by the one or more processors, the first speed of the motor to the hold speed in response to receiving the input data.
13. The method of any one of the preceding embodiments, wherein determining the throttle setting to adjust operation of the motor comprises: comparing, by the one or more processors, the first difference to one or more threshold differences to select a threshold difference, each threshold difference associated with increases or decreases of energy to the motor; determining, by the one or more processors, an increase or decrease of energy to the motor based on the threshold difference; and determining, by the one or more processors, the throttle setting to adjust operation of the motor based on the increase or decrease of energy to the motor.
14. The method of any one of the preceding embodiments, further comprising: generating, by the one or more processors, an indicator to indicate a rotations per minute (RPM) value associated with the hold speed via a graphical user interface (GUI) on a user device; and rendering, by the one or more processors, the indicator using a display device of the user device.
15. The method of any one of the preceding embodiments, further comprising: in response to obtaining monitor data associated with the first speed or the second speed of the motor, generating, by the one or more processors, an indicator to indicate an RPM value associated with the first speed or the second speed via a graphical user interface (GUI) on a user device; and rendering, by the one or more processors, the indicator using a display device of the user device.
16. The method of any one of the preceding embodiments, further comprising: receiving, by the one or more processors, user input data associated with a plurality of user inputs indicating positions of the throttle system to determine a set of calibration throttle settings; in response to receiving the plurality of user inputs indicating the positions of the throttle system, obtaining, by the one or more processors, a plurality of motor sensor signals associated with a plurality of sensed positions of the throttle system; configuring, by the one or more processors, the set of calibration throttle settings based on the plurality of user inputs indicating the positions of the throttle system and the plurality of sensed positions of the throttle system; and calibrating, by the one or more processors, the throttle setting based on the first difference to adjust operation of the motor to reduce the first difference with the set of calibration throttle settings.
17. The method of any one of the preceding embodiments, wherein a first position in the plurality of user inputs indicating positions corresponds to a position where the throttle system is at its widest angle; and a second position in the plurality of user inputs indicating positions corresponds to a position where the throttle system is at its narrowest angle.
18. The method of any one of the preceding embodiments, wherein the motor comprises a first motor, and further comprising: determining, by the one or more processors, that the first motor is associated with a first model that was used to calibrate the throttle setting; in response to obtaining an indication that the first motor was replaced with a second motor, determining, by the one or more processors, that at least one previous set of calibration throttle settings for the first model are associated with the second motor; and if at least one previous set of calibration throttle settings satisfies this criteria, retrieving, by the one or more processors, a previous set of calibration throttle settings associated with the first model, and configure the set of calibration throttle settings to the previous set of calibration throttle settings.
19. One or more non-transitory, computer-readable mediums storing instructions recorded thereon that, when executed by a data processing apparatus, cause the data processing apparatus to perform operations comprising those of any of embodiments 1-18.
20. A system comprising one or more processors and memory storing instructions that, when executed by the processors, cause the processors to effectuate operations comprising those of any of embodiments 1-18.
21. A system comprising means for performing any of embodiments 1-18.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Some embodiments of the present disclosure are described herein in connection with a threshold. As described herein, satisfying a threshold can refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

Embodiments implemented in computer software (e.g., computer programs, computer program products, etc.) can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or codes on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes or instructions on a non-transitory processor-readable medium or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

What is claimed is:

1. A system for monitoring fluid in storage tanks to operate a pump, the system comprising:
one or more processors configured to:
obtain sensor data associated with a fluid level measured in a storage tank;
compare the fluid level measured in the storage tank with a threshold fluid level representing a minimum level at which a pump can operate safely;
in response to comparing the fluid level with the threshold fluid level, determine that the fluid level satisfies the threshold fluid level; and
provide a control signal that is configured to cause the pump to operate based on the fluid level satisfying the threshold fluid level.

2. The system of claim 1, wherein the one or more processors are further configured to:
in response to determining that the fluid level satisfies the threshold fluid level, generate a warning via a graphical user interface (GUI), the warning indicating that the fluid level in the storage tank is below a safe operation level; and
render the warning via the GUI at a device.

3. The system of claim 1, wherein the one or more processors are further configured to:
in response to determining that the fluid level satisfies the threshold fluid level, generate a warning control signal; and
provide the warning control signal to a device to initiate operation of a haptic feedback system of the device.

4. The system of claim 1, wherein the sensor data comprises first sensor data, the one or more processors further configured to:
obtain second sensor data associated with a measurement of dissolved solids in fluid stored in the storage tank;
compare the measurement of the dissolved solids with a dissolved solids threshold to determine that the dissolved solids threshold is satisfied;
in response to determining that the dissolved solids threshold is satisfied, generate an indicator via a GUI, the indicator indicating that the dissolved solids threshold is satisfied; and
render the indicator via a GUI of a device.

5. The system of claim 4, wherein the one or more processors are configured to:
generate the indicator to indicate a part per million (PPM) level of the dissolved solids in the fluid.

6. The system of claim 1, wherein the one or more processors configured to provide the control signal are configured to:
provide a sensor signal to a motor indicating low pressure of the fluid, the sensor signal configured to cause the motor coupled to the pump to change operation states.

7. The system of claim 6, wherein the one or more processors configured to provide the control signal are configured to:
provide the control signal to a fuel control system to adjust a rate of flow of fuel into the motor from a first rate to a second rate, the second rate associated with an idle state of the motor.

8. The system of claim 7, wherein the sensor data comprises first sensor data, the one or more processors further configured to:
obtain second sensor data associated with a second fluid level measured in the storage tank at a point in time after the first sensor data is generated;
determine that the second fluid level does not satisfy the threshold fluid level; and
provide a second control signal that is configured to cause the fuel control system to adjust the rate of flow of fuel into the motor, from the second rate to the first rate.

9. A method, comprising:
obtaining, by one or more processors, sensor data associated with a fluid level measured in a storage tank;
comparing, by the one or more processors, the fluid level measured in the storage tank with a threshold fluid level representing a minimum level at which a pump can operate safely;
in response to comparing the fluid level with the threshold fluid level, determining, by one or more processors, that the fluid level satisfies the threshold fluid level; and
providing, by the one or more processors, a control signal that is configured to cause the pump to operate based on the fluid level satisfying the threshold fluid level.

10. The method of claim 9, further comprising:
in response to determining that the fluid level satisfies the threshold fluid level, generating, by the one or more processors, a warning via a GUI, the warning indicating that the fluid level in the storage tank is below a safe operation level; and
rendering the warning via a GUI of a device.

11. The method of claim 9, further comprising:
in response to determining that the fluid level satisfies the threshold fluid level, generating, by one or more processors, a warning control signal; and
providing, by one or more processors, the warning control signal to a device to initiate operation of a haptic feedback system of the device.

12. The method of claim 9, wherein the sensor data comprises first sensor data, further comprising:
obtaining, by one or more processors, second sensor data associated with a measurement of dissolved solids in fluid stored in the storage tank;
comparing, by one or more processors, the measurement of the dissolved solids with a dissolved solids threshold to determine that the dissolved solids threshold is satisfied;
in response to determining that the dissolved solids threshold is satisfied, generating, by one or more processors, an indicator via a GUI indicating that the dissolved solids threshold is satisfied; and
rendering the indicator via the GUI of a device.

13. The method of claim 12, wherein generating the GUI comprises:
generating, by the one or more processors, the GUI to indicate a part per million (PPM) level of the dissolved solids in the fluid;
wherein the one or more processors configured to cause the GUI to be displayed are configured to:
providing, by the one or more processors, GUI data associated with the GUI to a device that is configured to receive the GUI data and generate a user interface in accordance with the GUI.

14. The method of claim 9, wherein providing control signal comprises:
providing, by the one or more processors, a sensor signal to a motor indicating low pressure of fluid, the sensor signal configured to cause the motor coupled to the pump to change operation states.

15. The method of claim 9, wherein providing control signal comprises:
providing, by the one or more processors, the control signal to a fuel control system to adjust a rate of flow of fuel into a motor to transition from a first rate to a second rate, the second rate associated with an idle state of the motor.

16. The method of claim 15, wherein the sensor data comprises first sensor data, comprising:
obtaining, by the one or more processors, second sensor data associated with a second fluid level measured in the storage tank at a point in time after the first sensor data is generated;
determining, by the one or more processors, that the second fluid level does not satisfy the threshold fluid level; and
providing, by the one or more processors, a second control signal that is configured to cause the fuel control system to adjust the rate of flow of fuel into the motor to transition from the second rate to the first rate.

17. A non-transitory computer-readable medium storing instructions there on that, when executed by one or more processors, cause the one or more processors to:
obtain sensor data associated with a fluid level measured in a storage tank;
compare the fluid level measured in the storage tank with a threshold fluid level representing a minimum level at which a pump can operate safely;
in response to comparing the fluid level with the threshold fluid level, determine that the fluid level satisfies the threshold fluid level; and
provide control data associated with a control signal that is configured to cause the pump to operate based on the fluid level satisfying the threshold fluid level.

18. The non-transitory computer-readable medium of claim 17, wherein the instructions further cause the one or more processors to:
in response to determining that the fluid level satisfies the threshold fluid level, generating a warning via a GUI, the warning indicating that the fluid level in the storage tank is below a safe operation level; and
render the warning via GUI of a device.

19. The non-transitory computer-readable medium of claim 17, wherein the instructions further cause the one or more processors to:
in response to determining that the fluid level satisfies the threshold fluid level, generating a warning control signal; and
provide the warning control signal to a device to initiate operation of a haptic feedback system of the device.

20. The non-transitory computer-readable medium of claim 17, wherein the sensor data comprises first sensor data, and wherein the instructions further cause the one or more processors to:
obtain second sensor data associated with a measurement of dissolved solids in fluid stored in the storage tank;
compare the measurement of the dissolved solids with a dissolved solids threshold to determine that the dissolved solids threshold is satisfied;
in response to determining that the dissolved solids threshold is satisfied, generate an indicator via a GUI, the indicator indicating that the dissolved solids threshold is satisfied; and
render the indicator via a GUI of a device.
